(19) United States
(12) Patent Application Publication
(10) Pub. No.: US 2016/0192847 A1
(43) Pub. Date: Jul. 7, 2016

(54) BIOLOGICAL INFORMATION MEASURING MODULE AND BIOLOGICAL INFORMATION MEASURING APPARATUS

(71) Applicant: SEIKO EPSON CORPORATION, Tokyo (JP)

(72) Inventor: Akira INAGAKI, Matsumoto-shi (JP)

(21) Appl. No.: 14/987,371

(22) Filed: Jan. 4, 2016

(30) Foreign Application Priority Data
Jan. 5, 2015 (JP) 2015-000107

(51) Int. Cl. A61B 5/024; A61B 5/02; A61B 5/04; A61B 5/022; A61B 5/00

(52) U.S. Cl. CPC A61B 5/02427; A61B 5/022; A61B 5/6824; A61B 5/04; A61B 5/02

(57) ABSTRACT

A biological information measuring apparatus includes a sensor unit as a biological information measuring module including a light emitting unit that emits light to an object, and a light receiving unit that receives light from the object. A circumference length of the light emitting unit on the outer circumference is equal to or greater than 1.9 mm and equal to or less than 9.5 mm.

### CROSS-REFERENCES TO RELATED APPLICATIONS

This application claims priority to Japanese Patent Application No. 2015-000107, filed Jan. 5, 2015, the entirety of which is hereby incorporated by reference.

### BACKGROUND

1. Technical Field

The present invention relates to a biological information measuring module, and a biological information measuring apparatus mounted with the biological information measuring module.

2. Related Art

Hitherto, there have been known measuring apparatuses that are worn around body parts, such as a wrist, by a band or the like and measure biological information such as a wearer's pulse waves, and wristwatch type electronic apparatuses having a function of measuring the biological information. For example, JP-A-2000-254105 discloses an arm mounted measuring apparatus which is worn around the arm (wrist) of a wearer (test subject) and is mounted with a biological information measuring module that measures biological information, such as pulse waves, using an optical pulse wave detection sensor.

Such apparatuses (measuring apparatus, electronic apparatus) optically measure the flow of blood under a skin surface and convert the measured blood flow into a signal to thereby obtain biological information such as pulse waves, and thus a configuration of a dimensional relationship between a light emitting unit and a light receiving unit becomes significantly important. For example, when the light receiving unit and the light emitting unit become larger to a certain degree, the accuracy of measurement deteriorates. On the other hand, when the light receiving unit and the light emitting unit become excessively larger, the apparatus becomes larger, which results in a problem of portability deterioration such as a burden to wearing a unit around an arm (wrist).

When such apparatuses (measuring apparatus, electronic apparatus) are used for purposes related to, for example, sports, portability and reductions in size and weight are significantly important viewpoints for preventing the worn apparatuses from affecting the performance of wearers (test subjects). In addition, for example, even when the apparatuses are used for medical and health purposes, consideration for avoiding imposing a burden to patients or wearers (test subjects) is required, and thus portability and reductions in size and weight are significantly important viewpoints. In this manner, apparatuses that are worn around parts, such as a wrist, to thereby obtain biological information are required to rigorously seek an improvement in portability and a reduction in size and weight.

However, in the arm mounted measuring apparatus disclosed in JP-A-2000-254105, there is no description regarding the size of the optical pulse wave detection sensor (dimensions pertaining to the light emitting unit and the light receiving unit), and a problem in a configuration of a dimensional relationship between the above-mentioned light emitting unit and light receiving unit is not mentioned.

### SUMMARY

An advantage of some aspects of the invention is to solve at least a part of the problems described above, and the invention can be implemented as the following forms or application examples.

### Application Example 1

A biological information measuring module according to this application example includes a light emitting unit that emits light to an object, and a light receiving unit that receives light which is reflected by the object. A circumference length of the light emitting unit on the outer circumference is equal to or greater than 1.9 mm and equal to or less than 9.5 mm.

According to the biological information measuring module, when the circumference length of the light emitting unit on the outer circumference becomes smaller than 1.9 mm, light emission intensity is insufficient, and thus the light intensity of the reflected light, received by the light receiving unit, which is necessary for detection cannot be secured, which results in a deterioration in the measurement accuracy of biological information. In addition, when the circumference length of the light emitting unit on the outer circumference exceeds 9.5 mm, an installation space for the light emitting unit becomes larger, and the biological information measuring module or a biological information measuring apparatus mounted with the biological information measuring module becomes larger accordingly, which may degrade portability.

According to this application example, the circumference length of the light emitting unit on the outer circumference which does not cause such disadvantages is set, and thus it is possible to realize a reduction in size while securing light emission intensity, to accurately measure biological information, and to provide the biological information measuring module having excellent portability.

### Application Example 2

In the biological information measuring module according to the application example, it is preferable that a circumference length of the light emitting unit on the outer circumference is equal to or greater than 2.5 mm and equal to or less than 8.0 mm.

According to this application example, it is possible to further increase light emission intensity and to provide the small-sized biological information measuring module.

### Application Example 3

In the biological information measuring module according to the application example, it is preferable that a circumference length of the light emitting unit on the outer circumference is equal to or greater than 3.0 mm and equal to or less than 5.0 mm.

According to this application example, it is possible to secure light emission intensity more sufficiently and to provide the biological information measuring module of which the size is further reduced.

### Application Example 4

In this case, provided is a biological information measuring module including a light emitting unit that emits light to an object, and a light receiving unit that receives light which is reflected by the object. A circumference length of the light receiving unit on the outer circumference is equal to or greater than 5.3 mm and equal to or less than 11.7 mm.

According to the biological information measuring module, when the circumference length of the light receiving unit on the outer circumference becomes smaller than 5.3 mm, a light receiving region becomes excessively narrow, and thus it is not possible to sufficiently receive light necessary for detection, which results in a deterioration in the measurement accuracy of biological information. In addition, when the circumference length of the light receiving unit on the outer circumference exceeds 11.7 mm, an installation space for the light receiving unit becomes larger, and the biological information measuring module itself or a biological information measuring apparatus mounted with the biological information measuring module may become larger accordingly, which may degrade portability.

According to this application example, the circumference length of the light receiving unit on the outer circumference which does not cause such disadvantages is set, and thus it is possible to realize a reduction in size while securing the amount of light received which is necessary for detection, to accurately measure biological information, and to provide the biological information measuring module having excellent portability.

### Application Example 5

In the biological information measuring module according to the application example, it is preferable that a circumference length of the light receiving unit on the outer circumference is equal to or greater than 5.8 mm and equal to or less than 11.0 mm.

According to this application example, it is possible to further increase the amount of light received by the light receiving unit and to provide the small-sized biological information measuring module.

### Application Example 6

In the biological information measuring module according to the application example, it is preferable that a circumference length of the light receiving unit on the outer circumference is equal to or greater than 6.8 mm and equal to or less than 9.0 mm.

According to this application example, it is possible to secure a more sufficient amount of light received, to improve measurement accuracy, and to provide the biological information measuring module of which the size is further reduced.

### Application Example 7

A biological information measuring module according to this application example includes a light emitting unit that emits light to an object, and a light receiving unit that receives light which is reflected by the object. An area of the light emitting unit is equal to or greater than 2.5 mm² and equal to or less than 5.0 mm².

According to the biological information measuring module, when the area of the light emitting unit becomes smaller than 2.5 mm², light emission intensity is insufficient, and thus the light intensity of the reflected light, received by the light receiving unit, which is necessary for detection cannot be secured, which results in a deterioration in the detection accuracy of biological information. In addition, when the area of the light emitting unit exceeds 5.0 mm², an installation space for the light emitting unit becomes larger, and the biological information measuring module itself or a biological information measuring apparatus mounted with the biological information measuring module becomes larger accordingly, which may degrade portability.

According to this application example, the area of the light emitting unit which does not cause such disadvantages is set, and thus it is possible to realize a reduction in size while securing light emission intensity, to accurately measure biological information, and to provide the biological information measuring module having excellent portability.

### Application Example 8

In the biological information measuring module according to the application example, it is preferable that the area of the light emitting unit is equal to or greater than 3.0 mm² and equal to or less than 4.6 mm².

According to this application example, it is possible to further increase light emission intensity and to provide the small-sized biological information measuring module.

### Application Example 9

In the biological information measuring module according to the application example, it is preferable that the area of the light emitting unit is equal to or greater than 3.3 mm² and equal to or less than 4.0 mm².

According to this application example, it is possible to secure light emission intensity more sufficiently and to provide the biological information measuring module of which the size is further reduced.

### Application Example 10

A biological information measuring module according to this application example includes a light emitting unit that emits light to an object, and a light receiving unit that receives light which is reflected by the object. An area of the light receiving unit is equal to or greater than 1.7 mm² and equal to or less than 8.5 mm².

According to the biological information measuring module, when the area of the light receiving unit becomes smaller than 1.7 mm², a light receiving region becomes excessively narrow, and thus it is not possible to sufficiently receive light necessary for detection, which results in a deterioration in the detection accuracy of biological information. In addition, when the area of the light receiving unit exceeds 8.5 mm², an installation space for the light receiving unit becomes larger, and the biological information measuring module itself or a biological information measuring apparatus mounted with the biological information measuring module may become larger accordingly, which may degrade portability.

According to this application example, the area of the light receiving unit which does not cause such disadvantages is set, and thus it is possible to realize a reduction in size while securing the amount of light received which is necessary for detection, to accurately measure biological information, and to provide the biological information measuring module having excellent portability.

### Application Example 11

In the biological information measuring module according to the application example, it is preferable that the area of the light receiving unit is equal to or greater than 2.3 mm² and equal to or less than 6.3 mm².

According to this application example, it is possible to further increase the amount of light received by the light receiving unit and to provide the small-sized biological information measuring module.

### Application Example 12

In the biological information measuring module according to the application example, it is preferable that the area of the light receiving unit is equal to or greater than 3.0 mm² and equal to or less than 4.0 mm².

According to this application example, in the light receiving unit, a more sufficient amount of light received can be secured, and thus measurement accuracy is improved, and it is possible to provide the biological information measuring module of which the size is further reduced.

### Application Example 13

In the biological information measuring module according to the application example, it is preferable that a plurality of the light emitting units are provided.

According to this application example, the plurality of light emitting units are provided, and thus light emission intensity can be secured more sufficiently. In addition, biological information is detected by detecting light beams from the plurality of light emitting units, and thus it is possible to configure the biological information measuring module having further improved measurement accuracy.

### Application Example 14

In the biological information measuring module according to the application example, it is preferable that the light receiving unit and the plurality of light emitting units are disposed so as to be lined up in a row in a plan view when seen from a vertical direction of a light receiving surface of the light receiving unit.

According to this application example, since the light receiving unit and the light emitting units are disposed in a row in a plan view when seen from a vertical direction of the light receiving surface of the light receiving unit, dead space is reduced, and thus it is possible to achieve space saving and to configure the biological information measuring module having a smaller size.

### Application Example 15

In the biological information measuring module according to the application example, it is preferable that the plurality of the light emitting units include a first light emitting unit and a second light emitting unit and the light receiving unit is disposed between the first light emitting unit and the second light emitting unit.

According to this application example, dead space is reduced, and thus it is possible to achieve space saving. In addition, light beams from both the first light emitting unit and the second light emitting unit gather in the light receiving unit, and thus it is possible to perform detection more accurately.

### Application Example 16

In the biological information measuring module according to the application example, it is preferable that the plurality of the light emitting units are disposed at line symmetrical positions with respect to a virtual line passing through the center of the light receiving unit.

According to this application example, since the light emitting unit is disposed at a line symmetrical position with respect to the light receiving unit, dead space is reduced, and thus it is possible to achieve space saving. In addition, light beams from both the first light emitting unit and the second light emitting unit, which are located at line symmetrical positions, gather in the light receiving unit, and thus it is possible to perform detection more accurately.

### Application Example 17

In the biological information measuring module according to the application example, it is preferable that a reflective functional layer that reflects light emitted from the light emitting unit is provided in at least a portion of a vicinity of the light emitting unit.

According to this application example, light emitted from a peripheral direction of the light emitting unit can be made to be reflected by a reflective functional layer and to be directed to an object. Thereby, it is possible to increase the intensity (light emission intensity) of light directed to the object and to stabilize the measurement accuracy of biological information.

### Application Example 18

In the biological information measuring module according to the application example, it is preferable that an optical filter film is provided in a light receiving region of the light receiving unit.

According to this application example, it is possible to provide the optical filter in a smaller region and to provide the small-sized biological information measuring module.

### Application Example 19

In the biological information measuring module according to the application example, it is preferable that a light shielding unit is provided between the light emitting unit and the light receiving unit.

According to this application example, disturbance light or stray light of reflected light can be blocked by a light shielding unit such as a light shielding wall, and thus it is possible to perform detection (measurement) more accurately.

### Application Example 20

A biological information measuring apparatus according to this application example includes the biological information measuring module according to any one of the above-mentioned application examples.

According to this application example, detection (measurement) can be performed more accurately, and the biological information measuring module having a small size and excellent portability is provided, and thus it is possible to stably detect biological information and to provide the biological information measuring apparatus having a small size and excellent portability.

### BRIEF DESCRIPTION OF THE DRAWINGS

The invention will be described with reference to the accompanying drawings, wherein like numbers reference like elements.

FIGS. 1A and 1B are perspective views illustrating the exterior of a biological information measuring apparatus according to a first embodiment.

FIG. 2 is a side view illustrating the exterior of the biological information measuring apparatus of the first embodiment.

FIG. 3 is a diagram illustrating the wearing of the biological information measuring apparatus and communication with a terminal device.

FIG. 4 is a functional block diagram of the biological information measuring apparatus.

FIGS. 5A and 5B illustrate a sensor unit as a biological information measuring module, FIG. 5A is a front cross-sectional view, and FIG. 5B is a plan view seen from line A-A.

FIGS. 6A and 6B are graphs illustrating the suitability of circumference lengths of a light emitting unit and a light receiving unit on the outer circumferences.

FIGS. 7A and 7B are graphs illustrating the suitability of areas of the light emitting unit and the light receiving unit.

FIG. 8 is a plan view illustrating Modification Example 1 of the arrangement of a light emitting unit and a light receiving unit.

FIG. 9 is a plan view illustrating Modification Example 2 of the arrangement of a light emitting unit and a light receiving unit.

FIG. 10 is a cross-sectional view illustrating an example of the art of a biological information measuring apparatus according to a second embodiment.

FIG. 11 is a perspective view illustrating the biological information measuring apparatus according to the second embodiment.

FIG. 12 is a front view illustrating a biological information measuring apparatus according to a third embodiment.

FIG. 13 is a perspective view illustrating a biological information measuring apparatus according to a fourth embodiment.

FIG. 14 is a cross-sectional view illustrating a biological information measuring apparatus according to a fifth embodiment.

FIG. 15 is a flow chart illustrating a method of manufacturing the biological information measuring apparatus according to the second to fifth embodiments.

FIG. 16 is a schematic diagram illustrating a web page serving as a starting point of a health manager in a biological information measuring apparatus according to a sixth embodiment.

FIG. 17 is a diagram illustrating an example of a nutrition web page.

FIG. 18 is a diagram illustrating an example of an activity level web page.

FIG. 19 is a diagram illustrating an example of a mental concentration web page.

FIG. 20 is a diagram illustrating an example of a sleep web page.

FIG. 21 is a diagram illustrating an example of a daily activity web page.

FIG. 22 is a diagram illustrating an example of a health degree web page.

FIG. 23 is a partial cross-sectional view illustrating a modification example of a light receiving unit.

FIG. 24 is a partial cross-sectional view illustrating a modification example of a light emitting unit.

### DESCRIPTION OF EXEMPLARY EMBODIMENTS

Hereinafter, this embodiment will be described. Meanwhile, this embodiment described below does not improperly limit the contents of the invention which are described in the appended claims. In addition, all of the components described in this embodiment are not necessarily essential components of the invention.

### First Embodiment

### 1. Overall Configuration Example of Biological Information Measuring Apparatus

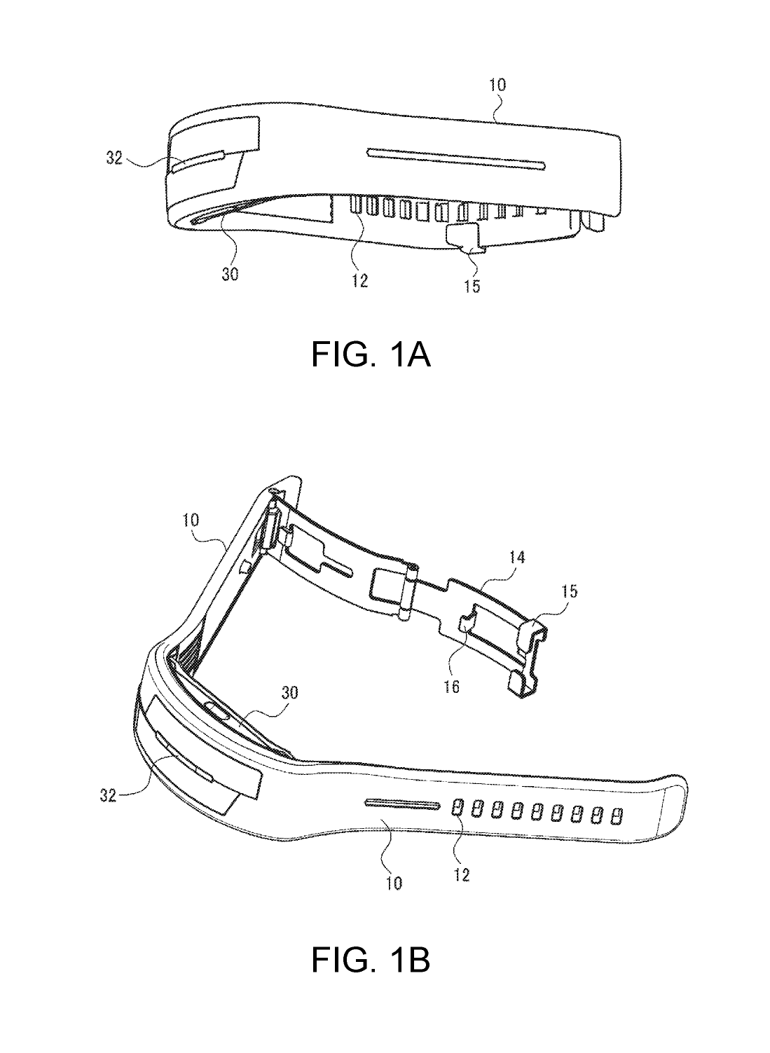

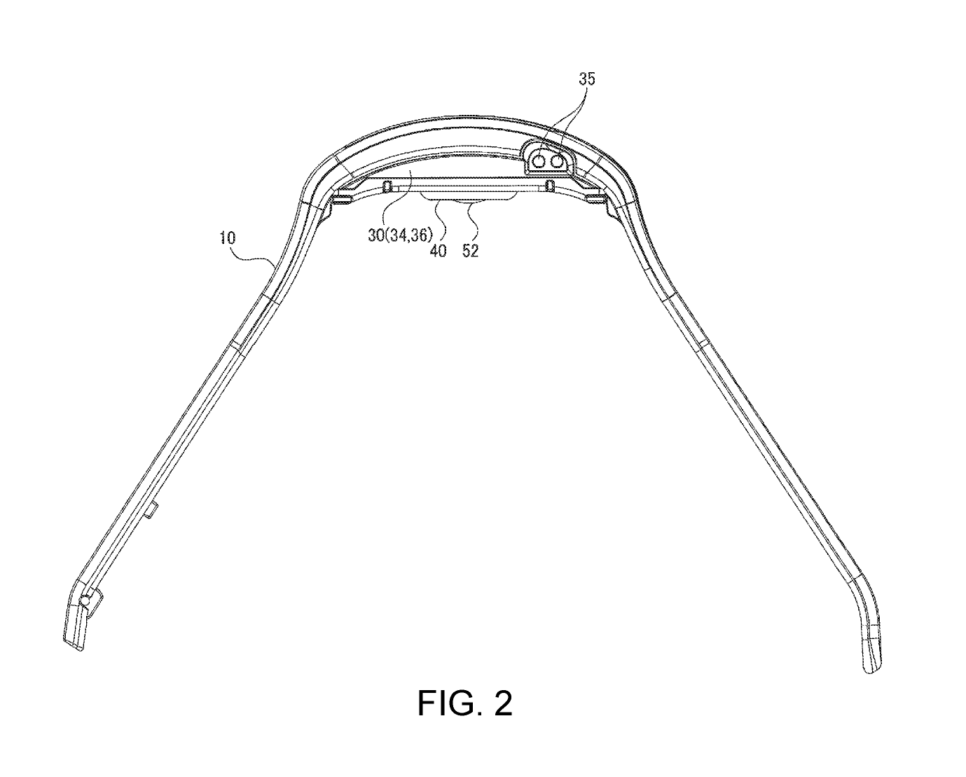

FIGS. 1A and 1B and FIG. 2 are schematic diagrams illustrating the exterior of a biological information measuring apparatus (biological information detecting apparatus) according to a first embodiment. FIG. 1A is a diagram when the biological information measuring apparatus is seen from the front, FIG. 1B is a diagram when the biological information measuring apparatus of FIG. 1A is obliquely seen from above, and FIG. 2 is a diagram when the biological information measuring apparatus is seen from the side.

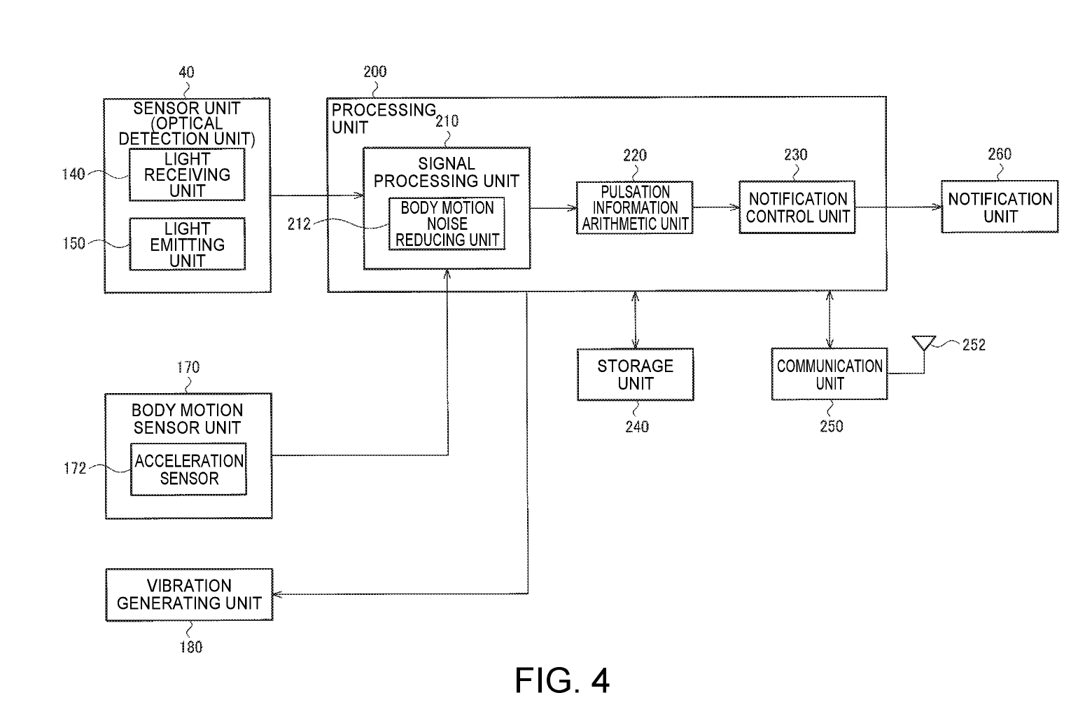

As illustrated in FIGS. 1A and 1B and FIG. 2, the biological information measuring apparatus of this embodiment includes a band portion 10, a case portion 30, and a sensor unit 40 as a biological information measuring module. The case portion 30 is attached to the band portion 10. The sensor unit 40 is provided in the case portion 30. In addition, the biological information measuring apparatus includes a processing unit 200 as illustrated in FIG. 4 to be described later. The processing unit 200 is provided in the case portion 30, and detects biological information on the basis of a detection signal from the sensor unit 40. Meanwhile, the biological information measuring apparatus of this embodiment is not limited to the configurations illustrated in FIG. 1A and FIG. 1B and FIG. 2, and various modifications such as the omission of some of the components thereof, replacement with other components, or the addition of other components can be made.

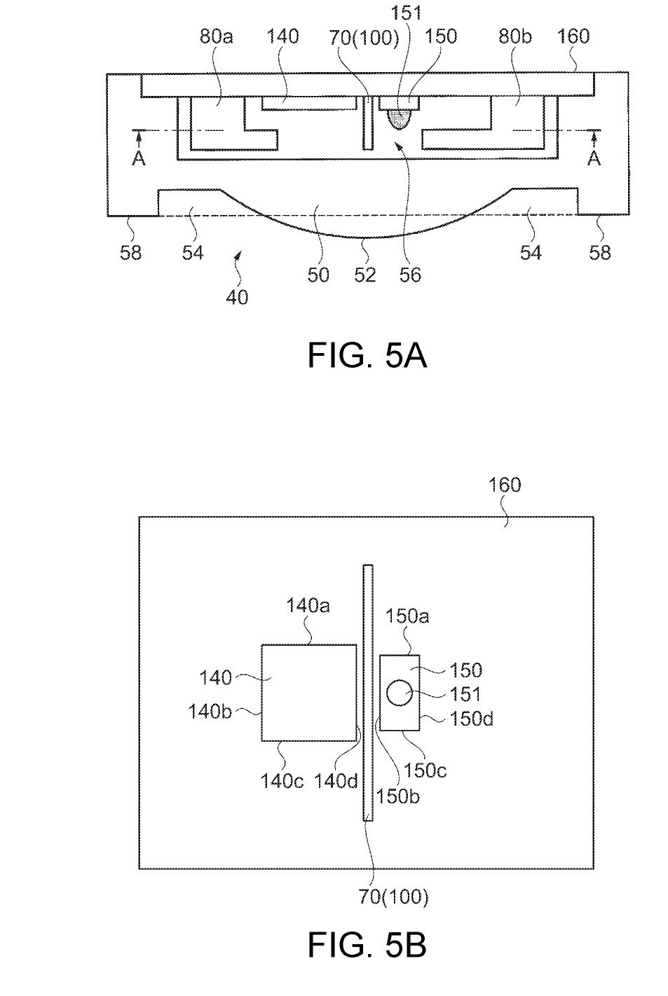

The sensor unit 40 as a biological information measuring module includes a substrate 160, a light emitting unit 150, a light receiving unit 140, a light shielding member 70, a light detection unit including a throttle portion 80 (80a, 80b), and other members, as described later with reference to FIGS. 5A and 5B. In the example illustrated in FIGS. 5A and 5B, the other members include a convex portion 52, a groove portion 54, a concave portion 56, a pressing suppressing portion 58, and the like which are realized by the light transmitting member 50. Here, a modification can also be made in which the light detection unit according to this embodiment includes these members, that is, the entire sensor unit 40 corresponds to the light detection unit.

Referring back to FIGS. 1A and 1B and FIG. 2, the band portion 10 is wound around the wrist of a wearer (hereinafter, also referred to as a user) so that the biological information measuring apparatus is worn thereon. The band portion 10 includes band holes 12 and a buckle portion 14. The buckle portion 14 includes a band insertion portion 15 and a protrusion portion 16. The user inserts one end side of the band portion 10 into the band insertion portion 15 of the buckle portion 14 and inserts the protrusion portion 16 of the buckle portion 14 into the band hole 12 of the band portion 10 to thereby wear the biological information measuring apparatus around his or her wrist. In this case, the magnitude of pressing (pressing against the surface of the wrist) by the sensor unit 40 to be described later is adjusted according to into which of the band holes 12 the protrusion portion 16 is inserted.

The case portion 30 is equivalent to a main body portion of the biological information measuring apparatus. Various components of the biological information measuring apparatus such as the sensor unit 40 and the processing unit 200 (see FIG. 4) are provided within the case portion 30. That is, the case portion 30 is a housing that accommodates the components. The case portion 30 includes, for example, a top case 34 which is positioned on the opposite side to the wrist and a bottom case 36 which is positioned on the wrist side. Meanwhile, the case portion 30 may not be configured so as to separate into the top case 34 and the bottom case 36.

The case portion 30 is provided with a light emitting window portion 32. The light emitting window portion 32 is formed of a light transmitting member. In addition, the case portion 30 is provided with a light emitting unit (LED, a light emitting unit for a notice which is different from the light emitting unit 150 of the light detection unit) which is mounted on a flexible substrate, and light from the light emitting unit is emitted to the outside of the case portion 30 through the light emitting window portion 32.

As illustrated in FIG. 2, the case portion 30 is provided with a terminal portion 35. When the biological information measuring apparatus is mounted on a cradle not shown in the drawing, a terminal portion of the cradle and the terminal portion 35 of the case portion 30 are electrically connected to each other. Thereby, a secondary battery (battery) provided in the case portion 30 can be charged.

The sensor unit 40 as a biological information measuring module detects biological information such as, for example, pulse waves of a test subject. For example, the sensor unit 40 includes a light receiving unit 140 and a light emitting unit 150 as illustrated in FIG. 4 and FIGS. 5A and 5B to be described later. In addition, the sensor unit 40 is formed of the light transmitting member 50 and includes the convex portion 52 that comes into contact with a test subject's skin surface and applies pressure. In this manner, the light emitting unit 150 emits light in a state where the convex portion 52 applies pressure to the skin surface, the light receiving unit 140 receives the light reflected by the test subject (blood vessel), and the light reception result thereof is output to the processing unit 200 as a detection signal. In addition, the processing unit 200 detects biological information, such as pulse waves, on the basis of the detection signal from the sensor unit 40. Meanwhile, biological information to be detected by the biological information measuring apparatus of this embodiment is not limited to pulse waves (pulse rate), and the biological information measuring apparatus may be an apparatus that detects biological information (for example, oxygen saturation in the blood, body temperature, heartbeat, and the like) other than pulse waves.

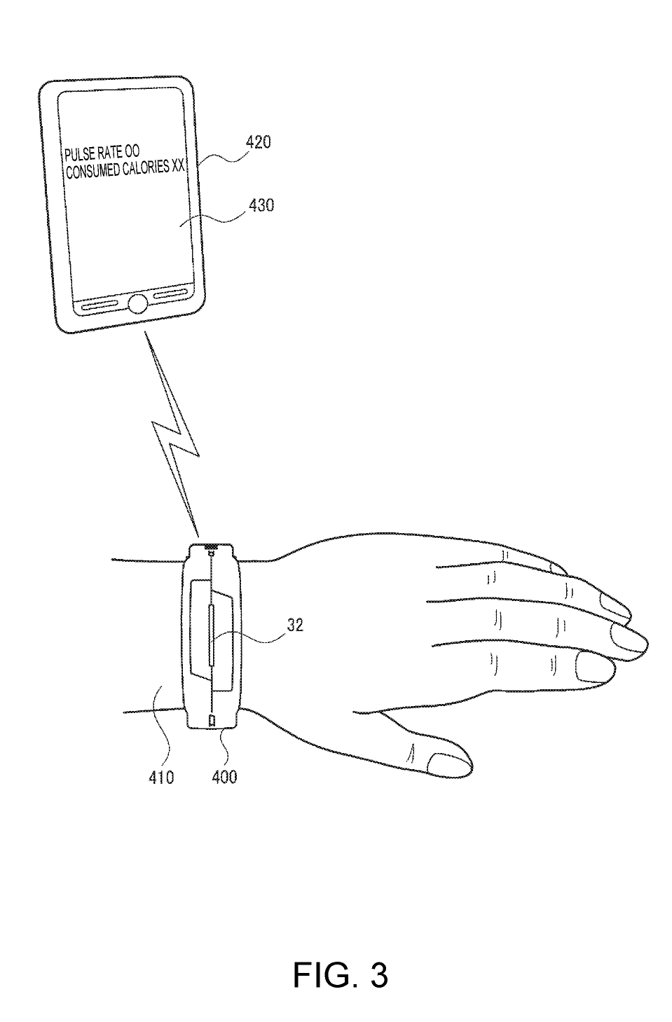

FIG. 3 is a schematic diagram illustrating the wearing of a biological information measuring apparatus 400 and communication with a terminal device 420. As illustrated in FIG. 3, a user who is a test subject wears the biological information measuring apparatus 400 around a wrist 410 like a wristwatch. As illustrated in FIG. 2, the sensor unit 40 is provided on a surface of the case portion 30 on the test subject side. Accordingly, when the biological information measuring apparatus 400 is worn, the convex portion 52 of the sensor unit 40 comes into contact with the skin surface of the wrist 410 and applies pressure. In this state, the light emitting unit 150 of the sensor unit 40 emits light, and the light receiving unit 140 receives the reflected light, and thus biological information such as pulse waves is detected.

The biological information measuring apparatus 400 and the terminal device 420 are connected to each other for communication, and thus data can be exchanged therebetween. The terminal device 420 is a portable communication terminal such as, for example, a smartphone, a mobile phone, or a feature phone. Alternatively, the terminal device 420 may be an information processing terminal such as a tablet computer. Proximity wireless communication such as, for example, Bluetooth (registered trademark) can be adopted as a communication connection between the biological information measuring apparatus 400 and the terminal device 420. In this manner, the biological information measuring apparatus 400 and the terminal device 420 are connected to each other for communication connection, and thus various pieces of information such as a pulse rate and consumed calories can be displayed on a display unit 430 (LCD or the like) of the terminal device 420. That is, various pieces of information obtained on the basis of the detection signal of the sensor unit 40 can be displayed. Meanwhile, the arithmetic processing of information such as a pulse rate or consumed calories may be performed by the biological information measuring apparatus 400, or at least a portion thereof may be performed by the terminal device 420.

The biological information measuring apparatus 400 is provided with the light emitting window portion 32, so that a user is notified of various pieces of information by light emission (lighting, blinking) of a light emitting body for a notice (not shown). For example, in the case of entering a fat combustion zone in information such as consumed calories or in the case of leaving the fat combustion zone, this is given notice of by the light emission of the light emitting body through the light emitting window portion 32. In addition, when an e-mail is received in the terminal device 420, the biological information measuring apparatus 400 is notified of the received e-mail from the terminal device 420. The light emitting body of the biological information measuring apparatus 400 emits light, and thus a user is notified of the reception of an e-mail or the like.

In this manner, in the example illustrated in FIG. 3, the biological information measuring apparatus 400 is not provided with a display unit such as an LCD, and thus information required to be given notice of by characters or numerals is displayed on the display unit 430 of the terminal device 420. In this manner, in the example illustrated in FIG. 3, a user is notified of the necessary minimum information by the light emission of the light emitting body without providing a display unit such as an LCD, thereby realizing a reduction in the size of the biological information measuring apparatus 400. In addition, the biological information measuring apparatus 400 is not provided with a display unit, and thus it is possible to improve the beauty of the biological information measuring apparatus 400.

FIG. 4 is a functional block diagram of the biological information measuring apparatus of this embodiment. The biological information measuring apparatus illustrated in FIG. 4 includes the sensor unit 40 as a biological information measuring module, a body motion sensor unit 170, a vibration generating unit 180, the processing unit 200, a storage unit 240, a communication unit 250, an antenna 252, and a notification unit 260. Meanwhile, the biological information measuring apparatus of this embodiment is not limited to the configuration illustrated in FIG. 4, and various modifications such as the omission of some of the components thereof, replacement with other components, or the addition of other components can be made.

The sensor unit 40 as a biological information measuring module detects biological information such as pulse waves, and includes the light receiving unit 140 and the light emitting unit 150. A pulse wave sensor (photoelectric sensor) is realized by the light receiving unit 140, the light emitting unit 150, and the like. The sensor unit 40 outputs a signal detected by the pulse wave sensor as a pulse wave detection signal.

The body motion sensor unit 170 outputs a body motion detection signal which is a signal varying in response to body motion, on the basis of pieces of sensor information of various sensors. The body motion sensor unit 170 includes, for example, an acceleration sensor 172 as a body motion sensor. Meanwhile, the body motion sensor unit 170 may include a pressure sensor, a gyro sensor, or the like as the body motion sensor.

The processing unit 200 performs various types of signal processes and control processes, for example, with the storage unit 240 as a work area, and can be realized by, for example, a processor such as a CPU or a logic circuit such as an ASIC. The processing unit 200 includes a signal processing unit 210, a pulsation information arithmetic unit 220, and a notification control unit 230.

The signal processing unit 210 performs various types of signal processes (filtering and the like), and performs signal processing on, for example, a pulse wave detection signal from the sensor unit 40, a body motion detection signal from the body motion sensor unit 170, or the like. For example, the signal processing unit 210 includes a body motion noise reducing unit 212. The body motion noise reducing unit 212 performs processing for reducing (removing) body motion noise which is noise caused by body motion, from the pulse wave detection signal, on the basis of the body motion detection signal from the body motion sensor unit 170. Specifically, the body motion noise reducing unit performs a noise reduction process using, for example, an adaptive filter.

The pulsation information arithmetic unit 220 performs arithmetic processing of pulsation information on the basis of a signal from the signal processing unit 210, and the like. The pulsation information is information such as, for example, a pulse rate. Specifically, the pulsation information arithmetic unit 220 obtains a spectrum by performing frequency analysis processing such as FFT on the pulse wave detection signal having been subjected to the noise reduction process by the body motion noise reducing unit 212, and performs a process of setting a representative frequency in the obtained spectrum as a frequency of a heartbeat. A value obtained by increasing the obtained frequency by 60 times is set to be a pulse rate (heart rate) which is generally used. Meanwhile, the pulsation information is not limited to the pulse rate itself, and may be various other pieces of information (for example, the frequency or cycle of a heartbeat) which indicate, for example, a pulse rate. In addition, the pulsation information may be information indicating the state of pulsation, or a value indicating, for example, the amount of blood itself may be set as pulsation information.

The notification control unit 230 controls the notification unit 260. The notification unit 260 (notification device) notifies a user of various pieces of information under the control of the notification control unit 230. For example, a light emitting body for a notice can be used as the notification unit 260. In this case, the notification control unit 230 controls a current flowing to an LED to thereby control the lighting, blinking, or the like of the light emitting body. Meanwhile, the notification unit 260 may be a display unit, such as an LCD, a buzzer, or the like.

In addition, the notification control unit 230 controls the vibration generating unit 180. The vibration generating unit 180 notifies a user of various pieces of information by vibration. The vibration generating unit 180 can be realized by, for example, a vibration motor (vibrator). The vibration motor generates vibration, for example, by rotating an eccentric weight. Specifically, the eccentric weight is attached to both ends of a driving shaft (rotor shaft) so that the motor itself shakes. The vibration of the vibration generating unit 180 is controlled by the notification control unit 230. Meanwhile, the vibration generating unit 180 is not limited to such a vibration motor, and various modifications can be made. The vibration generating unit 180 may be realized by, for example, a piezo element.

For example, a notice of start-up at the time of power-on, a notice of the first success in detecting pulse waves, a warning when a pulse-wave undetectable state is continued for a fixed period of time, a notice at the time of the movement of a fat combustion zone, a warning at the time of a battery voltage drop, a notice of a wake-up alarm, or a notice of an e-mail or a call from a terminal device such as a smartphone can be performed by the vibration of the vibration generating unit 180. Meanwhile, the pieces of information may be given notice of by a light emitting unit for a notice, or may be given notice of by both the vibration generating unit 180 and the light emitting unit.

The communication unit 250 performs communication with the external terminal device 420 as described in FIG. 3. For example, the communication unit performs wireless communication according to a standard such as Bluetooth (registered trademark). Specifically, the communication unit 250 receives a signal from the antenna 252 and transmits a signal to the antenna 252. The function of the communication unit 250 can be realized by a processor for communication or a logic circuit such as an ASIC.

### 2. Configuration Example of Sensor Unit as Biological Information Measuring Module

A configuration example of the sensor unit 40 as a biological information measuring module will be described below with reference to FIGS. 5A and 5B. FIGS. 5A and 5B are diagrams illustrating a detailed configuration example of the sensor unit 40. FIG. 5A is a front cross-sectional view, and FIG. 5B is a plan view seen from line A-A. Meanwhile, in FIG. 5B, the arrangement of the light receiving unit 140, the light emitting unit 150, and the light shielding member 70 (light shielding wall 100) as a light shielding unit is shown, and other components are not shown.

The sensor unit 40 as the biological information measuring module includes the light receiving unit 140 and the light emitting unit 150. The light receiving unit 140 and the light emitting unit 150 are mounted on the substrate 160 (sensor substrate) at a predetermined interval. The light emitting unit 150 emits light to an object (test subject or the like), and the light receiving unit 140 receives light (reflected light, transmitted light, or the like) from the object. For example, when the light emitting unit 150 emits light and the light is reflected by an object (for example, a blood vessel), the light receiving unit 140 receives the reflected light and detects. The light receiving unit 140 can be realized by a light receiving element such as, for example, a photodiode. The light emitting unit 150 can be realized by alight emitting element such as, for example, an LED. For example, the light receiving unit 140 can be realized by a diode element of a PN junction which is formed on a semiconductor substrate, or the like. In this case, an angle limiting filter for narrowing a light reception angle or a wavelength limiting filter (optical filter film) that limits a wavelength of light incident on a light receiving element may be formed on the diode element.

Meanwhile, a dome-type lens 151 (condensing lens in a broad sense) which is provided in the light emitting unit 150 is a lens for condensing light from an LED chip (light emitting element chip in a broad sense) which is resin-sealed (sealed with a light transmitting resin) in the light emitting unit 150. That is, in the light emitting unit 150 which is a surface-mounted type, the LED chip is disposed below the dome-type lens 151, and light from the LED chip is condensed by the dome-type lens 151 and is emitted to an object. Thereby, it is possible to improve the optical efficiency of the light detection unit.

When a pulsimeter is taken as an example of the biological information measuring apparatus, light from the light emitting unit 150 travels within a test subject which is an object, and is diffused or scattered to epidermis, dermis, subcutaneous tissue, and the like. Thereafter, the light reaches a blood vessel (part to be detected) and is reflected. At this time, a portion of the light is absorbed into the blood vessel. Since the absorption of the light at the blood vessel varies by the influence of pulses and the amount of reflected light also varies, the light receiving unit 140 receives the reflected light and detects variations in the amount of light, and thus it is possible to detect a pulse rate which is biological information, and the like.

Such a biological information measuring apparatus optically measures the blood flow in a skin surface and converts the blood flow into a signal to thereby obtain biological information such as pulse waves and pulses, and thus a configuration of a dimensional relationship between the light emitting unit 150 and the light receiving unit 140 becomes a significantly important element for the accuracy and stability of measurement. For example, when the light receiving unit 140 and the light emitting unit 150 become larger to a certain degree, the accuracy of measurement may deteriorate (deterioration in measurement accuracy). On the other hand, when the light receiving unit and the light emitting unit become excessively larger, the biological information measuring apparatus becomes larger, which results in a problem of a portability deterioration such as a burden to wearing around an arm (wrist).

Specifically, portability becomes a significantly important viewpoint from consideration for preventing the worn biological information measuring apparatus from affecting the performance of a wearer (test subject) when the apparatus is used for the purposes related to, for example, sport, or consideration for avoiding imposing a burden to a patient or a wearer (test subject) when the apparatus is used for medical and health purposes.

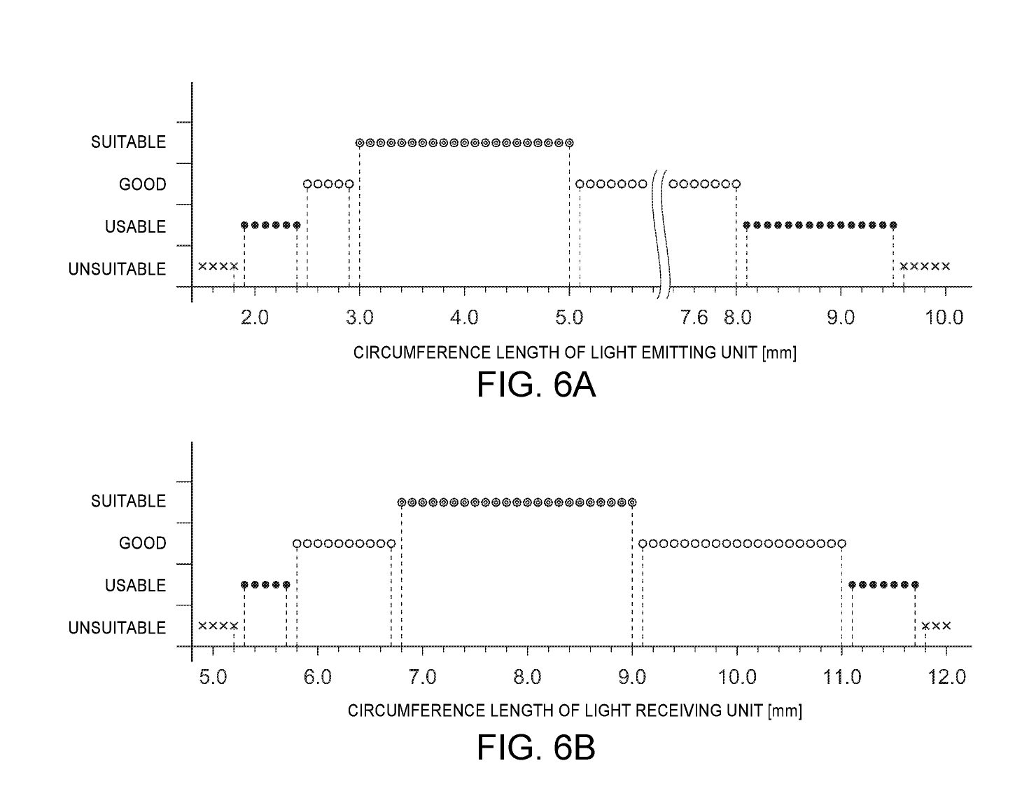

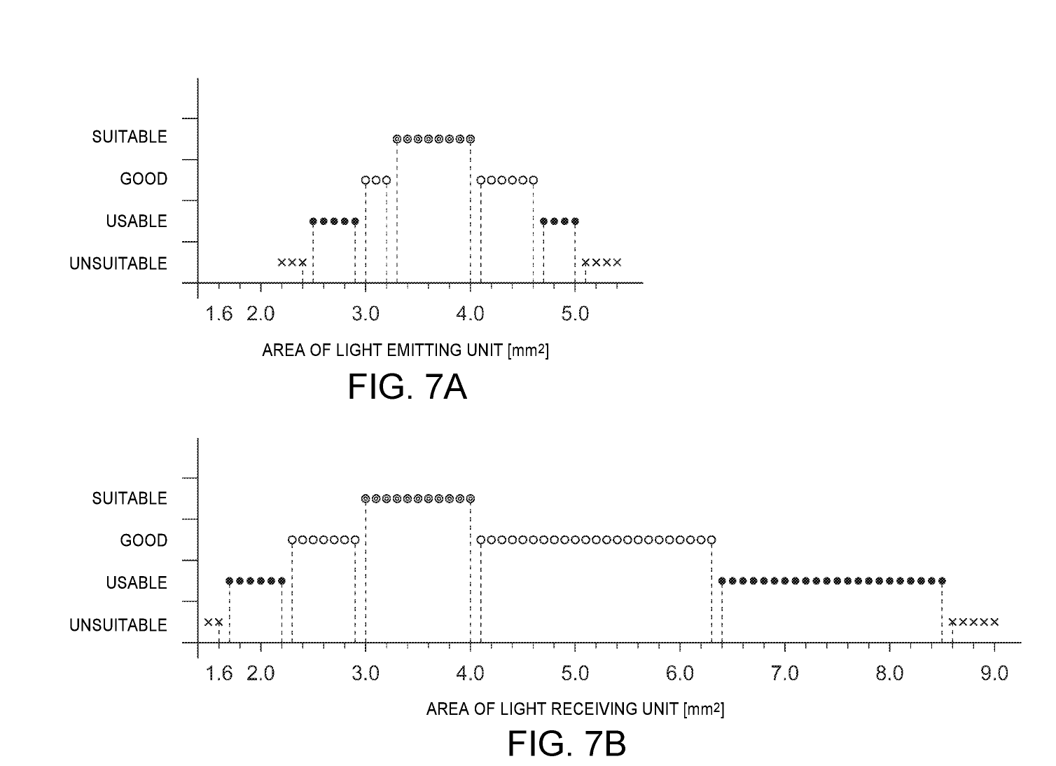

From such a viewpoint, the inventors have found a configuration of a dimensional relation which is excellent in portability while securing the accuracy and stability of measurement by wholeheartedly examining and verifying a configuration of a dimensional relationship between the light emitting unit 150 and the light receiving unit 140. Hereinafter, a preferable configuration of a dimensional relationship between the light emitting unit 150 and the light receiving unit 140 will be described with reference to FIGS. 6A and 6B and FIGS. 7A and 7B. Here, FIGS. 6A and 6B illustrate the suitability of circumference lengths of the light emitting unit and the light receiving unit. FIG. 6A is a graph illustrating verification results of suitability pertaining to a circumference length of the light emitting unit on the outer circumference, and FIG. 6B is a graph illustrating verification results of suitability pertaining to a circumference length of the light receiving unit on the outer circumference. FIGS. 7A and 7B illustrate the suitability of areas of the light emitting unit and the light receiving unit. FIG. 7A is a graph illustrating verification results of suitability pertaining to the area of the light emitting unit, and FIG. 6B is a graph illustrating verification results of suitability pertaining to the area of the light receiving unit.

### Circumference Length of Light Emitting Unit

First, a preferable configuration (range) of a circumference length of the light emitting unit 150 on the outer circumference will be described with reference to FIG. 6A. In the graph of FIG. 6A, a horizontal axis represents a circumference length of the light emitting unit 150 on the outer circumference, and verification results (determination results) of the suitability in the circumference lengths are shown. Meanwhile, the wording “the circumference length of the light emitting unit 150 on the outer circumference” as used herein refers to the total sum of a length dimension of a first side 150a constituting the outer circumference of the light emitting unit 150, a length dimension of a second side 150b, a length dimension of a third side 150c, and a length dimension of a fourth side 150d. Meanwhile, hereinafter, a description will be given in which the wording “circumference length of the light emitting unit 150 on the outer circumference” will be omitted and referred to as the wording “circumference length of the light emitting unit 150”.

The element size of the light emitting unit 150 increases as the circumference length of the light emitting unit 150 becomes larger, and the light emission intensity of light emitted from the light emitting unit 150 also becomes higher accordingly. Therefore, it is preferable to increase the circumference length of the light emitting unit 150 from the viewpoint of securing detection accuracy, but an excessive increase in the element size hinders a reduction in the size of the biological information measuring apparatus.

The circumference length of the light emitting unit 150 being equal to or less than 1.8 mm can make the size of the sensor unit 40 small and is suitable for a reduction in the size of the biological information measuring apparatus. However, the element size of the light emitting unit 150 is excessively reduced, which results in an insufficient light emission intensity of light to be emitted. For this reason, the amount of light received which is necessary for detection by the light receiving unit 140 becomes insufficient, which results in a disadvantage in that measurement cannot be accurately performed or the stability of measurement deteriorates. On the other hand, when the circumference length of the light emitting unit 150 is set to be equal to or greater than 9.6 mm, the element size of the light emitting unit 150 is excessively increased, and the size of the sensor unit 40 is also increased accordingly. Consequently, the biological information measuring apparatus becomes larger, and thus there is a concern for the occurrence of a disadvantage such as an uncomfortable feeling during the wearing thereof. Accordingly, as illustrated in FIG. 6A, when the circumference length of the light emitting unit 150 is equal to or less than 1.8 mm and the circumference length of the light emitting unit 150 is equal to or greater than 9.6 mm, it is determined that the light emitting unit is not suitable (unsuitable) for use. In other words, when the circumference length of the light emitting unit 150 is within a range from equal to or greater than 1.9 mm to equal to or less than 9.5 mm, the light emitting unit can be suitably used for the biological information measuring apparatus. A detailed description thereof will be given below.

Specifically, when the circumference length of the light emitting unit 150 is set to be equal to or greater than 1.9 mm, the light emission intensity of light to be emitted is increased, and thus it is possible to confirm that the amount of light received which is necessary for detection by the light receiving unit 140 can be secured. As a result, the measurement (detection) by the light receiving unit 140 becomes accurate, and the stability of the measurement can also be improved, and thus it can be determined that the light receiving unit is durable for use. Here, since the element size of the light receiving unit is small, it is effective to reduce the size of the sensor unit 40, but there is a manufacturing problem such as a deterioration in a non-defective rate (yield rate) or the necessity of more strictly performing manufacturing management (increase in the number of man-hours) in order to cope with the deterioration in a non-defective rate.

In addition, when the circumference length of the light emitting unit 150 is equal to or less than 9.5 mm, the sensor unit 40 mounted with the light emitting unit 150 has an allowable size. Consequently, the biological information measuring apparatus can be configured as a small-sized apparatus.

Further, when the circumference length of the light emitting unit 150 is set to be equal to or greater than 2.5 mm, the light emission intensity of light to be emitted is further increased, and thus it is possible to obtain a sufficient light emission intensity. Thereby, it is possible to more accurately perform measurement (detection) by the light receiving unit 140 and to further improve the stability of the measurement. Meanwhile, as the element size of the light emitting unit 150 increases, a manufacturing problem is further relieved but still remains slightly.

In addition, when the circumference length of the light emitting unit 150 is equal to or less than 8.0 mm, the element size of the light emitting unit 150 is significantly reduced, and thus it is possible to further reduce the size of the sensor unit 40 and to realize the small-sized biological information measuring apparatus.

In this manner, the circumference length of the light emitting unit 150 is set to be within a range from equal to or greater than 2.5 mm and equal to or less than 8.0 mm, and thus the light emitting unit 150 can be further suitably used for the biological information measuring apparatus.

Further, when the circumference length of the light emitting unit 150 is set to be equal to or greater than 3.0 mm, the light emission intensity of light to be emitted is further increased, and thus it is possible to obtain a sufficient light emission intensity. Thereby, it is possible to more accurately perform measurement (detection) by the light receiving unit 140 and to further improve the stability of the measurement. Meanwhile, in the element size in which the circumference length of the light emitting unit 150 is equal to or greater than 3.0 mm, a manufacturing problem is solved, and thus it is possible to improve a non-defective rate (yield rate) and to confirm that an extremely satisfactory state is set.

In addition, when the circumference length of the light emitting unit 150 is set to be equal to or less than 5.0 mm, the element size of the light emitting unit 150 is further reduced, and thus it is possible to reduce the size of the sensor unit 40 and to improve the arbitrariness of the arrangement of components constituting the sensor unit 40. Thereby, it is possible to realize the small-sized biological information measuring apparatus and to configure the biological information measuring apparatus of which the wearing can be maintained without causing an uncomfortable feeling even when, for example, an unexpected impact is applied thereto.

In this manner, the circumference length of the light emitting unit 150 is set to be within a range from equal to or greater than 3.0 mm and equal to or less than 5.0 mm, and thus the light emitting unit 150 can be particularly suitably used for the biological information measuring apparatus.

### Circumference Length of Light Receiving Unit

Next, a preferable configuration (range) of a circumference length of the light receiving unit 140 on the outer circumference will be described with reference to FIG. 6B. In the graph of FIG. 6B, a horizontal axis represents a circumference length of the light receiving unit 140 on the outer circumference, and verification results (determination results) of the suitability in the circumference lengths are shown. Meanwhile, the wording “the circumference length of the light receiving unit 140 on the outer circumference” as used herein refers to the total sum of a length dimension of a first side 140a constituting the outer circumference of the light receiving unit 140, a length dimension of a second side 140b, a length dimension of a third side 140c, and a length dimension of a fourth side 140d. Meanwhile, hereinafter, a description will be given in which the wording “circumference length of the light receiving unit 140 on the outer circumference” will be omitted and referred to as the wording “circumference length of the light receiving unit 140”.

The element size of the light receiving unit 140 increases as the circumference length of the light receiving unit 140 becomes larger, and a light receiving region also becomes larger accordingly. As the light receiving region becomes larger, it is possible to sufficiently receive light reflected from an object and to improve measurement (detection) accuracy. Therefore, it is preferable to increase the circumference length of the light receiving unit 140 from the viewpoint of securing detection accuracy, but an excessive increase in the element size results in an increase in the size of an installation space for the light receiving unit 140 and hinders a reduction in the size of the biological information measuring apparatus.

The circumference length of the light receiving unit 140 being equal to or less than 5.2 mm can make the size of the sensor unit 40 small and is suitable for a reduction in the size of the biological information measuring apparatus. However, a light receiving region becomes excessively narrow due to an excessive reduction in the element size of the light receiving unit 140, and thus light necessary for the detection cannot be sufficiently received, which results in a deterioration in the measurement accuracy of biological information. On the other hand, when the circumference length of the light receiving unit 140 is set to be equal to or greater than 11.8 mm, the element size of the light receiving unit 140 is excessively increased, and the size of the sensor unit 40 is also increased accordingly. Consequently, the biological information measuring apparatus becomes larger, and thus there is a concern for the occurrence of a disadvantage, such as an uncomfortable feeling, during the wearing thereof. Accordingly, as illustrated in FIG. 6B, when the circumference length of the light receiving unit 140 is equal to or less than 5.2 mm and the circumference length of the light receiving unit 140 is equal to or greater than 11.8 mm, it is determined that the light emitting unit is not suitable (unsuitable) for use. In other words, when the circumference length of the light receiving unit 140 is within a range from equal to or greater than 5.3 mm to equal to or less than 11.7 mm, the light receiving unit can be suitably used for the biological information measuring apparatus. A detailed description thereof will be given below.

Specifically, when the circumference length of the light receiving unit 140 is set to be equal to or greater than 5.3 mm, a light receiving region becomes larger, and thus it is possible to confirm that the amount of light received which is necessary for detection can be secured. As a result, the measurement (detection) by the light receiving unit 140 becomes accurate, and the stability of the measurement can also be improved, and thus it can be determined that the light receiving unit is durable for use. Here, since the element size of the light receiving unit 140 is small, it is effective to reduce the size of the sensor unit 40 similar to the light emitting unit 150, but there is a manufacturing problem such as a deterioration in a non-defective rate (yield rate) or the necessity of more strictly performing manufacturing management (increase in the number of man-hours) in order to cope with the deterioration in a non-defective rate.

In addition, when the circumference length of the light receiving unit 140 is equal to or less than 11.7 mm, the sensor unit 40 mounted with the light receiving unit 140 has an allowable size. Consequently, the biological information measuring apparatus can be configured as a small-sized apparatus.

Further, when the circumference length of the light receiving unit 140 is set to be equal to or greater than 5.8 mm, a light receiving region is further increased, and thus light can be sufficiently received. Accordingly, it is possible to more accurately perform measurement (detection) by the light receiving unit 140 and to further improve the stability of the measurement. Meanwhile, as the element size of the light receiving unit 140 increases, a manufacturing problem is further relieved but still remains slightly.

In addition, when the circumference length of the light receiving unit 140 is equal to or less than 11.0 mm, the element size of the light receiving unit 140 is significantly reduced, and thus it is possible to further reduce the size of the sensor unit 40 and to realize the small-sized biological information measuring apparatus.

In this manner, the circumference length of the light receiving unit 140 is set to be within a range from equal to or greater than 5.8 mm and equal to or less than 11.0 mm, and thus the light receiving unit 140 can be further suitably used for the biological information measuring apparatus.

Further, when the circumference length of the light receiving unit 140 is set to be equal to or greater than 6.8 mm, a light receiving region is further increased, and thus a more sufficient amount of light received can be secured. Accordingly, it is possible to more accurately perform measurement (detection) by the light receiving unit 140 and to further improve the stability of the measurement. Meanwhile, in the element size in which the circumference length of the light receiving unit 140 is equal to or greater than 5.8 mm, a manufacturing problem is solved, and thus it is possible to improve a non-defective rate (yield rate) and to confirm that an extremely satisfactory state is set.

In addition, when the circumference length of the light receiving unit 140 is set to be equal to or less than 9.0 mm, the element size of the light receiving unit 140 is further reduced, and thus it is possible to reduce the size of the sensor unit 40 and to improve the arbitrariness of the arrangement of components constituting the sensor unit 40. Thereby, it is possible to realize the small-sized biological information measuring apparatus and to configure the biological information measuring apparatus of which the wearing can be maintained without causing an uncomfortable feeling even when, for example, an unexpected impact is applied thereto.

In this manner, the circumference length of the light receiving unit 140 is set to be within a range from equal to or greater than 5.8 mm and equal to or less than 9.0 mm, and thus the light receiving unit 140 can be particularly suitably used for the biological information measuring apparatus.

### Area of Light Emitting Unit

Next, a preferable configuration (range) of the area of the light emitting unit 150 will be described with reference to FIG. 7A. In the graph of FIG. 7A, a horizontal axis represents the area of the light emitting unit 150, and verification results (determination results) of the suitability in the areas are shown. Meanwhile, the wording the area of the light emitting unit 150″ as used herein refers to the area of the surface of the light emitting unit 150 when the light emitting unit is seen from an object side.

The light emission intensity of light emitted from the light emitting unit 150 becomes higher as the area of the light emitting unit 150 becomes larger. Therefore, it is preferable to increase the area of the light emitting unit 150 from the viewpoint of securing detection accuracy, but an excessive increase in the area of the light emitting unit 150, that is, an excessive increase in the element size thereof hinders a reduction in the size of the biological information measuring apparatus.

The area of the light emitting unit 150 being equal to or less than 2.4 mm² can make the size of the sensor unit 40 small and is suitable for a reduction in the size of the biological information measuring apparatus. However, the element size of the light emitting unit 150 is excessively reduced, which results in an insufficient light emission intensity of light to be emitted. For this reason, the amount of light received which is necessary for detection by the light receiving unit 140 becomes insufficient, which results in a disadvantage in that measurement cannot be accurately performed or the stability of measurement deteriorates. On the other hand, when the area of the light emitting unit 150 is set to be equal to or greater than 5.1 mm², the element size of the light emitting unit 150 is excessively increased, and the size of the sensor unit 40 is also increased accordingly. Consequently, the biological information measuring apparatus becomes larger, and thus there is a concern for the occurrence of a disadvantage, such as an uncomfortable feeling, during the wearing thereof. Accordingly, as illustrated in FIG. 7A, when the area of the light emitting unit 150 is equal to or less than 2.4 mm² and the area of the light emitting unit 150 is equal to or greater than 5.1 mm², it is determined that the light emitting unit is not suitable (unsuitable) for use. In other words, when the area of the light emitting unit 150 is within a range from equal to or greater than 2.5 mm² to equal to or less than 5.0=², the light emitting unit can be suitably used for the biological information measuring apparatus. A detailed description thereof will be given below.

Specifically, when the area of the light emitting unit 150 is set to be equal to or greater than 2.5 mm², the light emission intensity of light to be emitted is increased, and thus it is possible to confirm that the amount of light received which is necessary for detection by the light receiving unit 140 can be secured. As a result, the measurement (detection) by the light receiving unit 140 becomes accurate, and the stability of the measurement can also be improved, and thus it can be determined that the light receiving unit is durable for use. Here, since the element size of the light receiving unit is small, it is effective to reduce the size of the sensor unit 40, but there is a manufacturing problem such as a deterioration in a non-defective rate (yield rate) or the necessity of more strictly performing manufacturing management (increase in the number of man-hours) in order to cope with the deterioration in a non-defective rate.

In addition, when the area of the light emitting unit 150 is equal to or less than 5.0 mm², the sensor unit 40 mounted with the light emitting unit 150 has an allowable size. Consequently, the biological information measuring apparatus can be configured as a small-sized apparatus.

Further, when the area of the light emitting unit 150 is set to be equal to or greater than 3.0 mm², the light emission intensity of light to be emitted is further increased, and thus it is possible to obtain a sufficient light emission intensity. Thereby, it is possible to more accurately perform measurement (detection) by the light receiving unit 140 and to further improve the stability of the measurement. Meanwhile, as the element size of the light emitting unit 150 increases, a manufacturing problem is further relieved but still remains slightly.

In addition, when the area of the light emitting unit 150 is equal to or less than 4.6 mm², the element size of the light emitting unit 150 is significantly reduced, and thus it is possible to further reduce the size of the sensor unit 40 and to realize the small-sized biological information measuring apparatus.

In this manner, the area of the light emitting unit 150 is set to be within a range from equal to or greater than 3.0 mm² and equal to or less than 4.6 mm², and thus the light emitting unit 150 can be further suitably used for the biological information measuring apparatus.

Further, when the area of the light emitting unit 150 is set to be equal to or greater than 3.3 mm², the light emission intensity of light to be emitted is further increased, and thus it is possible to obtain a sufficient light emission intensity. Thereby, it is possible to more accurately perform measurement (detection) by the light receiving unit 140 and to further improve the stability of the measurement. Meanwhile, in the element size in which the area of the light emitting unit 150 is equal to or greater than 3.3 mm², a manufacturing problem is solved, and thus it is possible to improve a non-defective rate (yield rate) and to confirm that an extremely satisfactory state is set.

In addition, when the area of the light emitting unit 150 is set to be equal to or less than 4.0 mm², the element size of the light emitting unit 150 is further reduced, and thus it is possible to reduce the size of the sensor unit 40 and to improve the arbitrariness of the arrangement of components constituting the sensor unit 40. Thereby, it is possible to realize the small-sized biological information measuring apparatus and to configure the biological information measuring apparatus of which the wearing can be maintained without causing an uncomfortable feeling even when, for example, an unexpected impact is applied thereto.

In this manner, the area of the light emitting unit 150 is set to be within a range from equal to or greater than 3.3 mm² and equal to or less than 4.0 mm², and thus the light emitting unit 150 can be particularly suitably used for the biological information measuring apparatus.

### Area of Light Receiving Unit

Next, a preferable configuration (range) of the area of the light receiving unit 140 will be described with reference to FIG. 7B. In the graph of FIG. 7B, a horizontal axis represents the area of the light receiving unit 140, and verification results (determination results) of the suitability in the areas are shown. Meanwhile, the wording “the area of the light receiving unit 140” as used herein refers to the area of the surface of the light receiving unit 140 when the light receiving unit is seen from an object side.

The element size of the light receiving unit 140 increases as the area of the light receiving unit 140 becomes larger, and a light receiving region becomes larger accordingly. When the light receiving region becomes larger, it is possible to sufficiently receive light reflected from an object side and to improve measurement (detection) accuracy. Therefore, it is preferable to increase the area of the light receiving unit 140 from the viewpoint of securing detection accuracy, but an excessive increase in the area of the light receiving unit 140, that is, an excessive increase in the element size thereof results in an increase in the size of an installation space for the light receiving unit 140 and hinders a reduction in the size of the biological information measuring apparatus.

The area of the light receiving unit 140 being equal to or less than 1.6 mm² can make the size of the sensor unit 40 small and is suitable for a reduction in the size of the biological information measuring apparatus. However, a light receiving region becomes excessively narrow due to an excessive reduction in the element size of the light receiving unit 140, and thus light necessary for the detection cannot be sufficiently received, which results in a deterioration in the measurement accuracy of biological information. On the other hand, when the area of the light receiving unit 140 is set to be equal to or greater than 8.6 mm², the element size of the light receiving unit 140 is excessively increased, and the size of the sensor unit 40 is also increased accordingly. Consequently, the biological information measuring apparatus becomes larger, and thus there is a concern for the occurrence of a disadvantage, such as an uncomfortable feeling, during the wearing thereof. Accordingly, as illustrated in FIG. 7B, when the area of the light receiving unit 140 is equal to or less than 1.6 mm² and the area of the light receiving unit 140 is equal to or greater than 8.6 mm², it is determined that the light receiving unit is not suitable (unsuitable) for use. In other words, when the area of the light receiving unit 140 is within a range from equal to or greater than 1.7 mm² to equal to or less than 8.5 mm², the light receiving unit can be suitably used for the biological information measuring apparatus. A detailed description thereof will be given below.

Specifically, when the area of the light receiving unit 140 is set to be equal to or greater than 1.7 mm², a light receiving region becomes larger, and thus it is possible to confirm that the amount of light received which is necessary for detection can be secured. As a result, the measurement (detection) by the light receiving unit 140 becomes accurate, and the stability of the measurement can also be improved, and thus it can be determined that the light receiving unit is durable for use. Here, since the element size of the light receiving unit 140 is small, it is effective to reduce the size of the sensor unit 40 similar to the light emitting unit 150, but there is a manufacturing problem such as a deterioration in a non-defective rate (yield rate) or the necessity of more strictly performing manufacturing management (increase in the number of man-hours) in order to cope with the deterioration in a non-defective rate.

In addition, when the area of the light receiving unit 140 is equal to or less than 8.5 mm², the sensor unit 40 mounted with the light receiving unit 140 has an allowable size. Consequently, the biological information measuring apparatus can be configured as a small-sized apparatus.

Further, when the area of the light receiving unit 140 is set to be equal to or greater than 2.3 mm², a light receiving region is further increased, and thus light can be sufficiently received. Accordingly, it is possible to more accurately perform measurement (detection) by the light receiving unit 140 and to further improve the stability of the measurement. Meanwhile, as the element size of the light receiving unit 140 increases, a manufacturing problem is further relieved but still remains slightly.

In addition, when the area of the light receiving unit 140 is equal to or less than 6.3 mm², the element size of the light receiving unit 140 is significantly reduced, and thus it is possible to further reduce the size of the sensor unit 40 and to realize the small-sized biological information measuring apparatus.

In this manner, the area of the light receiving unit 140 is set to be within a range from equal to or greater than 2.3 mm² and equal to or less than 6.3 mm², and thus the light receiving unit 140 can be further suitably used for the biological information measuring apparatus.

Further, when the area of the light receiving unit 140 is set to be equal to or greater than 3.0 mm², a light receiving region is further increased, and thus a more sufficient amount of light received can be secured. Accordingly, it is possible to more accurately perform measurement (detection) by the light receiving unit 140 and to further improve the stability of the measurement. Meanwhile, in the element size in which the area of the light receiving unit 140 is equal to or greater than 3.0 mm², a manufacturing problem is solved, and thus it is possible to improve a non-defective rate (yield rate) and to confirm that an extremely satisfactory state is set.

In addition, when the area of the light receiving unit 140 is set to be equal to or less than 4.0 mm², the element size of the light receiving unit 140 is further reduced, and thus it is possible to reduce the size of the sensor unit 40 and to improve the arbitrariness of the arrangement of components constituting the sensor unit 40. Thereby, it is possible to realize the small-sized biological information measuring apparatus and to configure the biological information measuring apparatus of which the wearing can be maintained without causing an uncomfortable feeling even when, for example, an unexpected impact is applied thereto.

In this manner, the area of the light receiving unit 140 is set to be within a range from equal to or greater than 3.0 mm² and equal to or less than 4.0 mm², and thus the light receiving unit 140 can be particularly suitably used for the biological information measuring apparatus.

A description will be given by referring back to FIGS. 5A and 5B. The light shielding member 70 (light shielding wall 100) as a light shielding unit is provided between the light receiving unit 140 and the light emitting unit 150. The light shielding member 70 (light shielding wall 100) prevents light from, for example, the light emitting unit 150 (direct light or the like) from being directly incident on the light receiving unit 140. The light shielding member 70 (light shielding wall 100) can be formed by, for example, sheet metal working. Meanwhile, an example of a material of the light shielding member 70 (light shielding wall 100) includes a resin such as rubber (including a natural resin and a synthetic resin) as a material other than a metal material.

The light shielding member 70 as a light shielding unit is a member for shielding light. In this embodiment, the light shielding member 70 is provided between the light receiving unit 140 and the light emitting unit 150 as the light shielding wall 100, and shields the light receiving unit 140. Meanwhile, the light shielding member 70 may be provided so as to cover a portion of the light receiving unit 140, and may be configured to shield light incident on the light receiving unit 140. It is possible to improve detection performance while preventing light from the light emitting unit 150 from being incident on the light receiving unit 140, by the light shielding member 70 (light shielding wall 100).

In addition, it is preferable to perform a reflection suppressing process on at least the surface of the light receiving unit 140 on the side of the light shielding member 70 (light shielding wall 100) as a light shielding unit. For example, the light shielding member 70 is configured to have a surface (inner surface or the like) having a predetermined color such as a black color so that the irregular reflection of light is prevented. Alternatively, the light shielding member 70 may be configured to have a surface having a moth-eye structure. For example, a concavo-convex structure having several tens to several hundreds of cycles is formed in the surface of the light shielding member so as to configure a reflection preventing structure. When such a reflection suppressing process is performed, it is possible to effectively suppress the occurrence of a situation in which, for example, reflected light on the surface of the light shielding member 70 changes to stray light and becomes a noise component of a detection signal.

The light receiving unit 140, the light emitting unit 150, and the light shielding member 70 (light shielding wall 100) as a light shielding unit are mounted on the substrate 160. The substrate 160 is, for example, a rigid substrate. The substrate 160 is provided with a terminal (not shown) for connection to a terminal (not shown) of a signal and a power supply of the light receiving unit 140 and a terminal (not shown) for connection to a signal and a power supply of an external main substrate. For example, the terminal of the light receiving unit 140 and the terminal of the substrate 160 are connected to each other by wire bonding or the like.

In addition, the sensor unit 40 is provided with the throttle portion 80 (80a, 80b). The throttle portion 80 narrows light from a test subject in a light path between the test subject and the sensor unit 40, and narrows light from the light emitting unit 150. In FIGS. 5A and 5B, the throttle portion 80 is provided between the light transmitting member 50 and the light emitting unit 150. Here, the throttle portion 80 may be provided between the light transmitting member 50 and a test subject or within the light transmitting member 50.

The light transmitting member 50 is provided on a surface of the biological information measuring apparatus which comes into contact with a test subject, and transmits light from the test subject. In addition, the light transmitting member 50 comes into contact with the test subject when biological information of the test subject is measured. For example, the convex portion 52 (detection window) of the light transmitting member 50 comes into contact with the test subject. Meanwhile, it is preferable that the shape of the surface of the convex portion 52 is a curved surface shape (spherical shape). However, the invention is not limited thereto, and various shapes can be adopted. In addition, the light transmitting member 50 may be a member capable of transmitting a wavelength of light from a test subject, and a transparent material or a colored material may be used.

The groove portion 54 for suppressing a pressing fluctuation or the like is provided in the vicinity of the convex portion 52 of the light transmitting member 50. In addition, when a surface of the light transmitting member 50 which is provided with the convex portion 52 is set to be a first surface, the light transmitting member 50 has the concave portion 56 at a position corresponding to the convex portion 52 in a second surface on the back side of the first surface. The light receiving unit 140, the light emitting unit 150, the light shielding member 70, and the throttle portion 80 are provided in a space of the concave portion 56.

In addition, the pressing suppressing portion 58 that suppresses pressing applied to a test subject (skin of a wrist) by the convex portion 52 is provided on a surface of the biological information measuring apparatus on a test subject side. In FIGS. 5A and 5B, the pressing suppressing portion 58 is provided so as to surround the convex portion 52 of the light transmitting member 50. The convex portion 52 protrudes toward the test subject side further than a pressing suppressing portion (pressing suppressing surface) 58.

It is possible to apply initial pressing for exceeding, for example, a vein vanishing point to a test subject by providing the convex portion 52. In addition, the pressing suppressing portion 58 for suppressing pressing applied to the test subject by the convex portion 52 is provided, and thus it is possible to minimally suppress a pressing fluctuation in a usage range in which the measurement of biological information is performed by the biological information measuring apparatus and to achieve a reduction in a noise component and the like. In addition, when the convex portion 52 protrudes from the pressing suppressing portion 58, the convex portion 52 comes into contact with the test subject and applies initial pressing, and then the pressing suppressing portion 58 comes into contact with the test subject, and thus it is possible to suppress pressing applied to the test subject by the convex portion 52. The wording “vein vanishing point” as used herein refers to a point in which a signal caused by a vein superimposed on a pulse wave signal vanishes or becomes smaller to the extent that the signal does not affect the measurement of pulse waves, when the convex portion 52 is brought into contact with the test subject and the strength of pressing is sequentially increased.

According to the above-mentioned configuration of the first embodiment, a configuration of a dimensional relationship between the light emitting unit 150 and the light receiving unit 140 is accurately set, and thus it is possible to maintain and improve light emission intensity and light reception sensitivity and to provide the biological information measuring apparatus having a small size and excellent portability while securing the accuracy and stability of measurement.

### Modification Example of Arrangement of Light Emitting Unit and Light Receiving Unit

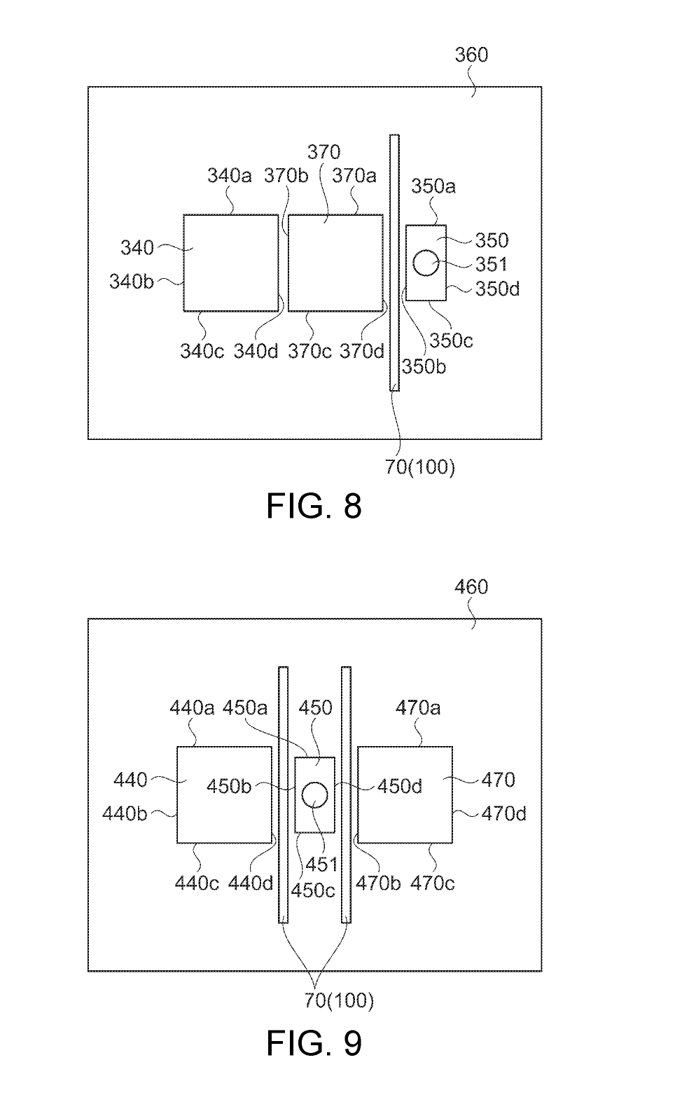

Next, a modification example of the arrangement of a light emitting unit and a light receiving unit will be described with reference to FIG. 8 and FIG. 9. FIG. 8 is a plan view illustrating Modification Example 1 of the arrangement of a light emitting unit and a light receiving unit. In addition, FIG. 9 is a plan view illustrating Modification Example 2 of the arrangement of a light emitting unit and a light receiving unit. Meanwhile, hereinafter, the same components as those in the above-described embodiment will be denoted by the same reference numerals and signs, and a description thereof may be omitted or simplified. In addition, in Modification Example 1 and Modification Example 2, the same configuration as that in the first embodiment can be applied to a dimensional configuration and a positional relationship such as circumference lengths of a light emitting unit 150 and a light receiving unit 140 or the areas thereof.

### Modification Example 1

Modification Example 1 of the arrangement of a light emitting unit and a light receiving unit will be described with reference to FIG. 8. In the first embodiment described above, one light emitting unit 150 and one light receiving unit 140 are mounted on the substrate 160 (sensor substrate) so as to be lined up. In a configuration of Modification Example 1, after a first light receiving unit 340 and a second light receiving unit 370 as light receiving units share a light emitting unit 350, the light emitting unit 350, the second light receiving unit 370, and the first light receiving unit 340 are mounted on a substrate 306 in this order so as to be lined up in a row along a predetermined direction.

In the case of Modification Example 1, the total dimension of a length dimension of a first side 350a constituting the outer circumference of the light emitting unit 350, a length dimension of a second side 350b, a length dimension of a third side 350c, and a length dimension of a fourth side 350d is equivalent to a circumference length of the light emitting unit 350 on the outer circumference. In addition, the total dimension of a length dimension of a first side 370a constituting the outer circumference of the second light receiving unit 370, a length dimension of a second side 370b, a length dimension of a third side 370c, and a length dimension of a fourth side 370d is equivalent to a circumference length of the light receiving unit on the outer circumference. Meanwhile, when the first light receiving unit 340 is adjacent to the light emitting unit 350, the total dimension of a length dimension of a first side 340a constituting the outer circumference of the first light receiving unit 340, a length dimension of a second side 340b, a length dimension of a third side 340c, and a length dimension of a fourth side 340d is equivalent to a circumference length of the light receiving unit on the outer circumference.

Meanwhile, the arrangement of the second light receiving unit 370 may be exchanged with the arrangement of the first light receiving unit 340. Specifically, the light emitting unit 350, the first light receiving unit 340, and the second light receiving unit 370 can be disposed in this order.

### Modification Example 2

Modification example 2 of the arrangement of a light emitting unit and a light receiving unit will be described with reference to FIG. 9. In a configuration of Modification Example 2, after a first light receiving unit 440 and a second light receiving unit 470 as light receiving units share a light emitting unit 450, the first light receiving unit 440 and the second light receiving unit 470 are mounted on both sides of the light emitting unit 450 so as to be lined up in a row along a predetermined direction.

In the case of Modification Example 2, the total dimension of a length dimension of a first side 450a constituting the outer circumference of the light emitting unit 450, a length dimension of a second side 450b, a length dimension of a third side 450c, and a length dimension of a fourth side 450d is equivalent to a circumference length of the light emitting unit 450 on the outer circumference. In addition, the total dimension of a length dimension of a first side 470a constituting the outer circumference of the second light receiving unit 470, a length dimension of a second side 470b, a length dimension of a third side 470c, and a length dimension of a fourth side 470d, or the total dimension of a length dimension of a first side 440a constituting the outer circumference of the first light receiving unit 440, a length dimension of a second side 440b, a length dimension of a third side 440c, and a length dimension of a fourth side 440d is equivalent to a circumference length of the light receiving unit on the outer circumference.

According to the configurations of Modification Example 1 and Modification Example 2, for example, the first light receiving unit can acquire a pulse signal, and the second light receiving unit can acquire a different signal such as a signal including a large amount of body motion noise. In this manner, when the second light receiving unit can detect a signal corresponding to body motion noise, it is possible to reduce body motion noise by removing (reducing) components corresponding to a detection signal in the second light receiving unit from a detection signal in the first light receiving unit. Thereby, in the configurations of Modification Example 1 and Modification Example 2, it is possible to acquire a pulse signal having reduced body motion noise and having a higher degree of accuracy, in addition to the effects of the first embodiment described above.

### Other Modification Examples

In addition, although not shown in the drawing, a configuration may also be adopted in which a plurality of light emitting units (two light emitting units in second to fifth embodiments) are provided, as illustrated in biological information measuring apparatuses according to the second to fifth embodiments, and the plurality of light emitting units and a light receiving unit are disposed so as to be lined up in a row. Regarding a circumference length of the light emitting unit on the outer circumference and the area thereof, at least one light emitting unit may correspond to such a configuration.

In this manner, in addition to the effects of the first embodiment described above, the plurality of light emitting units are provided, and thus it is possible to secure light emission intensity more sufficiently. In addition, biological information is detected by detecting light beams from the plurality of light emitting units, and thus it is possible to configure a biological information measuring apparatus with a further improved measurement accuracy.

### Second Embodiment

Next, the second embodiment of the invention will be described with reference to the accompanying drawings.

Similarly to the first embodiment described above, the biological information measuring apparatus (hereinafter, referred to as a measuring apparatus) according to the second embodiment is a heart rate monitoring apparatus which is worn on a living body (for example, a human body) of which biological information is measured, and which measures biological information such as a pulse (heart rate). Meanwhile, in the following drawings, each component has a size to the extent that the component can be recognized in the drawing, and thus a description may be given by appropriately making a dimension and proportion of each component different from those of an actual component.

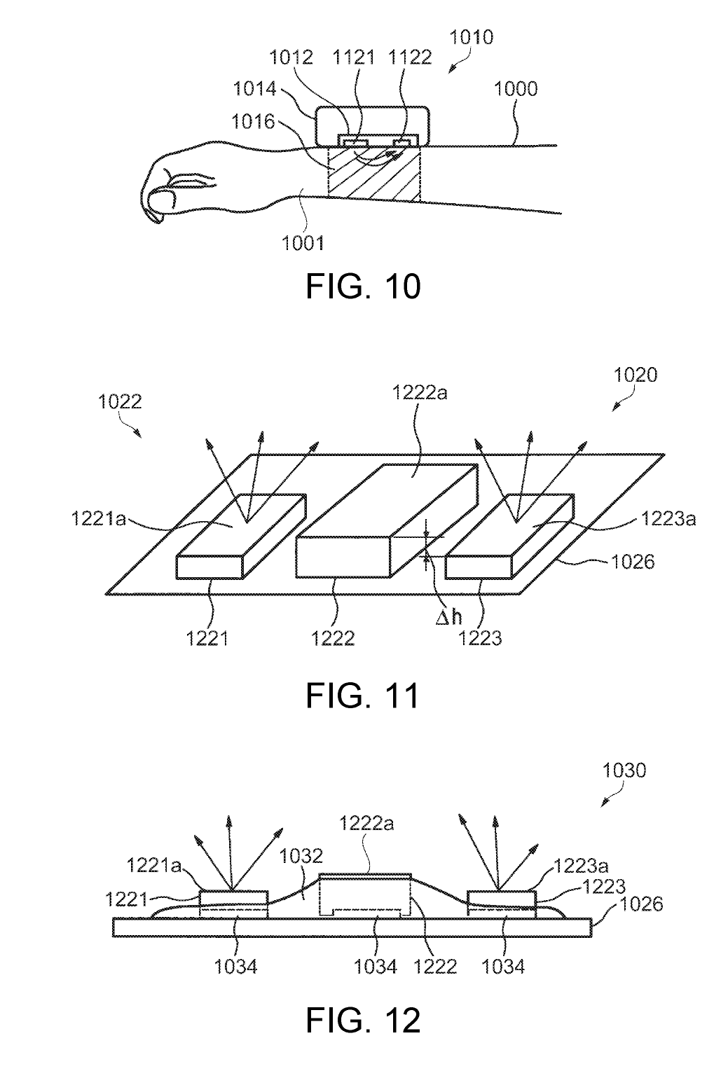

First, before a heart rate monitoring apparatus 1010 as the biological information measuring apparatus according to the second embodiment is described, an example of the related art of the heart rate monitoring apparatus as the biological information measuring apparatus according to the second embodiment will be described with reference to FIG. 10.

FIG. 10 is a cross-sectional view illustrating a heart rate monitoring apparatus 1010 as a biological information measuring apparatus according to an example of the related art which measures a physiologic parameter (biological information) of a user (test subject) 1000 (the user's arm is shown in the drawing) who is wearing the heart rate monitoring apparatus. The heart rate monitoring apparatus 1010 includes a sensor 1012 that measures a heart rate as at least one physiologic parameter of the user 1000, and a case 1014 that accommodates the sensor 1012. The heart rate monitoring apparatus 1010 is worn on the arm 1001 of the user 1000 by a fixation portion 1016 (for example, a band).

The sensor 1012 is a heart rate monitoring sensor that includes a light emitting element 1121 as a light emitting unit and a light receiving element 1122 as a light receiving unit which are two sensor elements and measures or monitors a heart rate. However, the sensor may be a sensor that measures one or more physiologic parameters (for example, a heart rate, blood pressure, the amount of air inhaled, skin conductivity, skin humidity, and the like). In addition, when the case 1014 includes a band-type housing, the heart rate monitoring apparatus can be used as a wristwatch type monitoring apparatus which is used in, for example, sport. Meanwhile, the case 1014 may have a shape capable of mainly holding the sensor 1012 at a desired position with respect to the user 1000, and may be able to arbitrarily accommodate more elements such as a battery, a processing unit, a display, and a user interface.

The biological information measuring apparatus of the conventional example is the heart rate monitoring apparatus 1010 for monitoring a user's heart rate. The sensor 1012 is an optical sensor constituted by the light emitting element 1121 and the light receiving element 1122. An optical heart rate monitor using the optical sensor depends on the light emitting element 1121 (LED is generally used) as a light source that exposes the skin to light. The light emitted from the light emitting element 1121 to the skin is partially absorbed by blood flowing through a blood vessel under the skin, but the rest of the light is reflected and leaves the skin. The reflected light is captured by the light receiving element 1122 (photodiode is generally used). A light reception signal from the light receiving element 1122 is a signal including information equivalent to the amount of blood flowing through the blood vessel. The amount of blood flowing through the blood vessel varies depending on pulse of the heart. In this manner, a signal on the light receiving element 1122 varies in response to the pulsation of the heart. In other words, a variation in the signal of the light receiving element 1122 is equivalent to the pulse of a heart rate. A pulse rate per unit time is counted (for example, per 10 seconds), to thereby obtain the number of beats of the heart for one minute (that is, a heart rate).

Hereinafter, a heart rate monitoring apparatus 1020 as the biological information measuring apparatus according to the second embodiment will be described with reference to FIG. 11. FIG. 11 is a perspective view illustrating a heart rate monitoring apparatus as the biological information measuring apparatus according to the second embodiment. Although not shown in FIG. 11, the heart rate monitoring apparatus 1020 as the biological information measuring apparatus according to the second embodiment is worn on a user's arm by a fixation portion such as a band, similar to the first embodiment described above.

In the heart rate monitoring apparatus 1020 as the biological information measuring apparatus according to the second embodiment, light emitting elements 1221 and 1223 as a plurality of (two in this example) light emitting units and a light receiving element 1222 as one light receiving unit are disposed so as to be lined up in a row. Specifically, a sensor 1022 (in this example, two light emitting elements 1221 and 1223 as a first light emitting unit and a second light emitting unit and the light receiving element 1222 as a light receiving unit are used as three sensor elements) which includes at least two sensor elements is provided.

The light receiving element 1222 as the light receiving unit is disposed between the two light emitting elements 1221 and 1223 as the first light emitting unit and the second light emitting unit. In addition, two light emitting elements 1221 and 1223 as the first light emitting unit and the second light emitting unit are disposed at line symmetrical positions with respect to a virtual line passing through the center of the light receiving element 1222 as the light receiving unit. The light emitting elements 1221 and 1223 and the light receiving element 1222 are disposed in such a manner, and thus it is possible to reduce a dead space and to achieve space saving. In addition, light beams from both the first light emitting unit and the second light emitting unit, which are located at line symmetrical positions, gather in the light receiving unit, and thus detection can be performed more accurately.

The sensor element detects a sensor signal. The sensor 1022 includes an optical sensor constituted by the light emitting elements 1221 and 1223 using two LEDs for emitting light to the skin of a user, and at least one light receiving element 1222 (photodiode) for receiving the light reflected from the skin. Further, the heart rate monitoring apparatus 1020 includes a case or a housing (not shown). The case or the housing may be similar to or the same as the case 1014 illustrated in FIG. 10, or may be similar to or the same as the case portion 30 in the first embodiment described above.

The sensor 1022 is carried on one surface of a carrier (substrate) 1026. Here, a configuration including the carrier (substrate) 1026 and the sensor 1022 carried on the carrier (substrate) 1026 corresponds to a biological information measuring module. Meanwhile, the same is true of the third to fifth embodiments. Light emitted from the light emitting elements 1221 and 1223 can be reflected without being absorbed into the skin or the like, and can directly reach the light receiving element 1222. In the heart rate monitoring apparatus 1020, a distance between the carrier 1026 and each of upper surfaces 1221a and 1223a of the respective light emitting elements 1221 and 1223 is smaller than a distance between the carrier 1026 and an upper surface 1222a of the light receiving element 1222. That is, a difference between the distance between the carrier 1026 and each of the upper surfaces 1221a and 1223a of the respective light emitting elements 1221 and 1223 and the distance between the carrier 1026 and an upper surface 1222a of the light receiving element 1222 is Δh. The light receiving element 1222 receives light from the upper surface 1222a thereof which is the uppermost surface layer. According to these configurations, there is an effect that the most of light emitted from the light emitting elements 1221 and 1223 is directed to the skin and reflected light is directly incident on the light receiving element 1222 without going through an air layer or the like. In other words, since a structure in which the light receiving element 1222 comes into close contact with the skin is formed, a structure in which a gap is not likely to be generated between the upper surface (light receiving surface) 1222a of the light receiving element 1222 and the skin can be formed, and thus it is possible to prevent light, such as external light, which serves as a noise source from being incident on the upper surface 1222a. In addition, light from the light emitting elements 1221 and 1223 which does not pass through the skin, for example, light being directly incident on the light receiving element 1222 from the light emitting elements 1221 and 1223 cannot reach the upper surface 1222a of the light receiving element 1222.

### Third Embodiment

Next, a heart rate monitoring apparatus 1030 as the biological information measuring apparatus according to the third embodiment will be described with reference to FIG. 12. FIG. 12 is a front view illustrating a heart rate monitoring apparatus as the biological information measuring apparatus according to the third embodiment. Meanwhile, although not shown in FIG. 12, the heart rate monitoring apparatus 1030 as the biological information measuring apparatus according to the third embodiment is worn on a user's arm by a fixation portion such as a band, similar to the first embodiment described above.

As illustrated in FIG. 12, electric connection terminals 1034 of light emitting elements 1221 and 1223 as light emitting units and a light receiving element 1222 as a light receiving unit have to be preferably covered with an insulating material (for example, epoxy resin) 1032 in order to protect electrical elements. In addition, a configuration can be adopted in which the insulating material 1032 does not cover the light emitting elements 1221 and 1223 and the light receiving element 1222. Specifically, a configuration can be adopted in which the insulating material 1032 is buried in a region between the light emitting element 1221 and the light receiving element 1222 and a region between the light emitting element 1223 and the light receiving element 1222. In other words, a configuration can be adopted in which at least an upper surface 1222a of the light receiving element 1222 and upper surfaces 1221a and 1223a of the light emitting elements 1221 and 1223 are not covered with the insulating material 1032. With such a configuration, it is possible to suppress disturbance due to an air gap between the skin and the light emitting elements 1221 and 1223. Further, a configuration may be adopted in which the insulating material 1032 covers the upper surfaces 1221a and 1223a of the light emitting elements 1221 and 1223 and the upper surface 1222a of the light receiving element 1222. With such a configuration, the upper surface 1222a of the light receiving element 1222 which comes into contact with the skin and the upper surfaces 1221a and 1223a of the light emitting elements 1221 and 1223 can be protected, and thus it is possible to prevent the upper surface 1222a of the light receiving element 1222 and the upper surfaces 1221a and 1223a of the light emitting elements 1221 and 1223 from being damaged. In this case, the insulating material 1032 can be regarded as a protection film.

In the heart rate monitoring apparatus 1030 as the biological information measuring apparatus according to this third embodiment, the insulating material 1032 using an epoxy resin is provided, as an example which is generally implementable. In FIG. 12, the insulating material 1032 is disposed so as not to cover the upper surfaces 1221a and 1223a of the light emitting elements 1221 and 1223, and protects the electric connection terminals 1034. Light beams emitted from the light emitting elements 1221 and 1223 are indicated by an arrow.

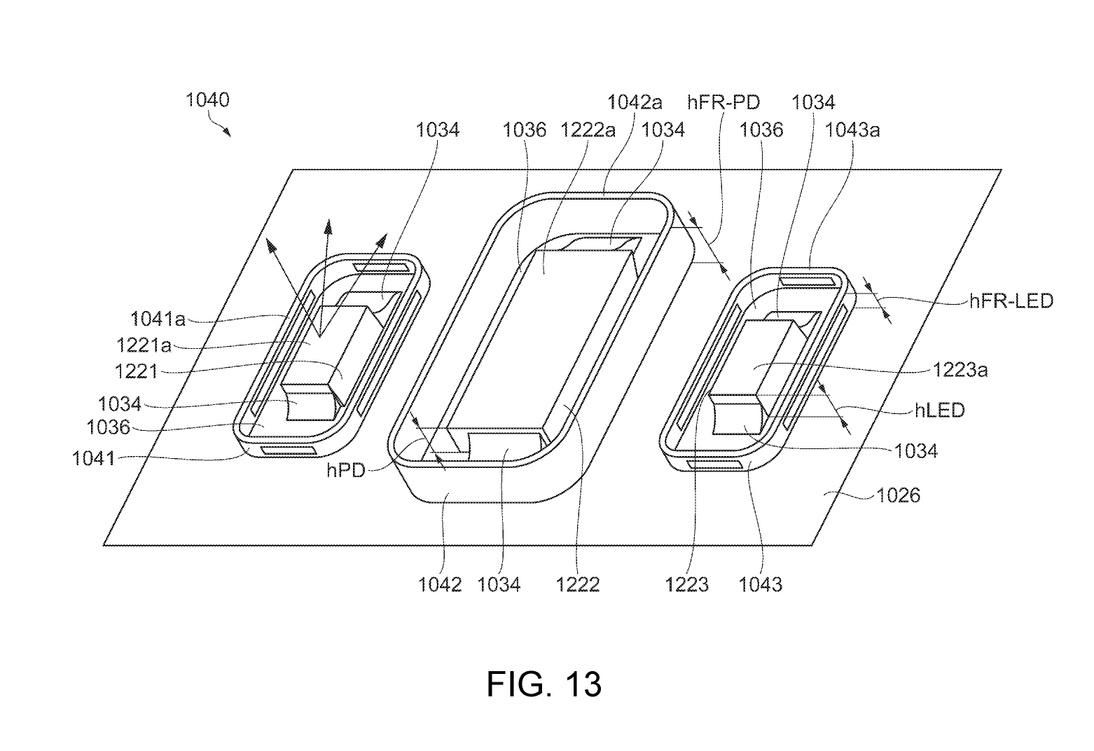

In this manner, the insulating material 1032 is minimally disposed to the extent that a correct function of the heart rate monitoring apparatus 1030 is not hindered, and thus the heart rate monitoring apparatus 1030 can be further improved by protecting the electric connection terminals 1034 of the light emitting elements 1221 and 1223 and the light receiving element 1222. Meanwhile, it is more preferable to configure a heart rate monitoring apparatus 1040 as the biological information measuring apparatus according to the fourth embodiment as illustrated in FIG. 13, instead of adopting the configuration of this third embodiment in which an epoxy resin is injected.

### Fourth Embodiment

Next, a heart rate monitoring apparatus 1040 as the biological information measuring apparatus according to the fourth embodiment will be described with reference to FIG. 13. FIG. 13 is a perspective view illustrating a heart rate monitoring apparatus as the biological information measuring apparatus according to the fourth embodiment. Meanwhile, although not shown in FIG. 13, the heart rate monitoring apparatus 1040 as the biological information measuring apparatus according to the fourth embodiment is worn on a user's arm by a fixation portion, such as a band, similar to the first embodiment described above.

In the heart rate monitoring apparatus 1040 as the biological information measuring apparatus according to the fourth embodiment, frames 1041, 1042, and 1043 created are disposed. The frames 1041, 1042, and 1043 are disposed in the vicinity of the light emitting elements 1221 and 1223 as light emitting units and the light receiving element 1222 as a light receiving unit, and a space 1036 is formed between each of the frames 1041, 1042, and 1043 and each of the light emitting elements 1221 and 1223 and the light receiving element 1222. An insulating material (not shown in FIG. 13) is injected with the frames 1041, 1042, and 1043 as guides to cover the electric connection terminals 1034 of the light emitting elements 1221 and 1223 and the light receiving element 1222.

In the example shown in the fourth embodiment, the light emitting elements 1221 and 1223 and the light receiving element 1222 are surrounded by the respective frames 1041, 1042, and 1043. Meanwhile, as another example, all of the frames 1041, 1042, and 1043 may be coupled to each other, or all of the sensor elements may be surrounded by an integrated frame. Meanwhile, the frames 1041, 1042, and 1043 can be used as light shielding walls as examples of light shielding units. The frames 1041, 1042, and 1043 are used as light shielding walls, and thus it is possible to prevent light emitted from the light emitting elements 1221 and 1223 from being directly incident on the light receiving element 1222.

As an improvement for preventing the function of the heart rate monitoring apparatus 1040 from being affected, it is preferable that upper edges 1041a and 1043a of the frames 1041 and 1043 in the vicinity of the light emitting elements 1221 and 1223 are lower than the upper surfaces 1221a and 1223a of the light emitting elements 1221 and 1223. In other words, a distance hFR-LED between the carrier 1026 and each of the upper edges 1041a and 1043a of the respective frames 1041 and 1043 is the same as or smaller than a distance hLED between the carrier 1026 and each of the upper surfaces 1221a and 1223a of the light emitting elements 1221 and 1223 which are surrounded by the respective frames 1041 and 1043 (hFR-LEDh≦LED). It is preferable that a difference between the distance hLED between the carrier 1026 and each of the upper surfaces 1221a and 1223a of the respective light emitting elements 1221 and 1223 and the distance hFR-LED between the carrier 1026 and each of the upper edges 1041a and 1043a of the respective frames 1041 and 1043 is set to be in a range from 0.1 mm to 0.8 mm. Meanwhile, it is more preferable that a difference between the distance hLED between the carrier 1026 and each of the upper surfaces 1221a and 1223a of the respective light emitting elements 1221 and 1223 and the distance hFR-LED between the carrier 1026 and each of the upper edges 1041a and 1043a of the respective frames 1041 and 1043 is set to be in a range from 0.2 mm to 0.5 mm.

In addition, it is preferable that an upper edge 1042a of the frame (receiver frame) 1042 in the vicinity of the light receiving element 1222 is higher than the upper surface 1222a of the light receiving element 1222. In other words, a distance hFR-PD between the carrier 1026 and the upper edge 1042a of the frame 1042 is larger than a distance hPD between the carrier 1026 and the upper surface 1222a of the light receiving element 1222 surrounded by the frame 1042 (hFR-PD>hPD).

It is preferable that a difference between the distance hPD between the carrier 1026 and the upper surface 1222a of the light receiving element 1222 and the distance hFR-PD between the carrier 1026 of the upper edge 1042a of the frame 1042 is set to be in a range from 0 mm to 0.5 mm. Meanwhile, it is more preferable that a difference between the distance hPD between the carrier 1026 and the upper surface 1222a of the light receiving element 1222 and the distance hFR-PD between the carrier 1026 and the upper edge 1042a of the frame 1042 is set to be in a range from 0.1 mm to 0.2 mm.

Further, the distance hFR-PD between the carrier 1026 and the upper edge 1042a of the frame 1042 is larger than the distance hLED between the carrier 1026 and the upper surfaces 1221a and 1223a of the respective light emitting elements 1221 and 1223 (hFR-PD>hLED).

Meanwhile, for example, when the light receiving element 1222 and the light emitting elements 1221 and 1223 are close to each other, a configuration may be adopted in which only one frame wall is present between the light receiving element 1222 and each of the light emitting elements 1221 and 1223. This may occur because of manufacturing easiness. When the one frame wall is a case, frame walls of the frames of both the light receiving element 1222 and each of the light emitting elements 1221 and 1223 are coincident with each other. This means that the frame walls of the light emitting elements 1221 and 1223 become relatively high. In detail, the frame wall on the light receiving element 1222 side out of the frame walls of the frames 1041 and 1043 surrounding the respective light emitting elements 1221 and 1223 becomes relatively high, and the other frame wall becomes lower than the upper surfaces 1221a and 1223a of the respective light emitting elements 1221 and 1223.

Further, instead of the frames 1041, 1042, and 1043, a configuration may be adopted in which a first wall portion is provided between the light receiving element 1222 and the light emitting element 1221 or the light emitting element 1223 and a second wall portion is provided on the outside of the light emitting elements 1221 and 1223, that is, on the side opposite to the first wall portion with respect to the light receiving element 1222. In such a configuration, a distance between the carrier 1026 and the upper surface of the first wall portion may be larger than a distance between the carrier 1026 and the upper surface of the second wall portion. With such a configuration, it is possible to realize the function of the frame using a smaller number of members than in a case where a light emitting element and a light receiving element are surrounded as illustrated in FIG. 13.

Meanwhile, the frames 1041 and 1043 and the frame 1042 are used as in this fourth embodiment, and thus it is possible to prevent an insulating material to be injected, such as an epoxy resin, from flowing out. In this manner, the partitioning of an insulating material such as an epoxy resin by creating an additional structure is option of allowing high mass productivity to be obtained. Meanwhile, the frames 1041 and 1043 and the frame 1042 may be formed of the same material as that of the carrier 1026. For example, the frames may be formed by injection molding using an epoxy-based resin or a polycarbonate-based resin.

As described above, the insulating material 1032 (see FIG. 12) protects the electric connection terminals 1034 of the sensor elements (light emitting elements 1221 and 1223 and the light receiving element 1222). However, the electric connection terminals 1034 have to further come into contact with additional electronic apparatuses (for example, a driver, detection electronics, a processor, or a power supply) which are other elements. This means that there is any electrical connection between the carrier 1026 (may be a printed circuit board (PCB)) and the additional electronic apparatuses. In addition, the structure of the heart rate monitoring apparatus according to this embodiment can be applied not only to an apparatus for measuring a heart rate but also to apparatuses for measuring pulse waves and pulse.

### Fifth Embodiment

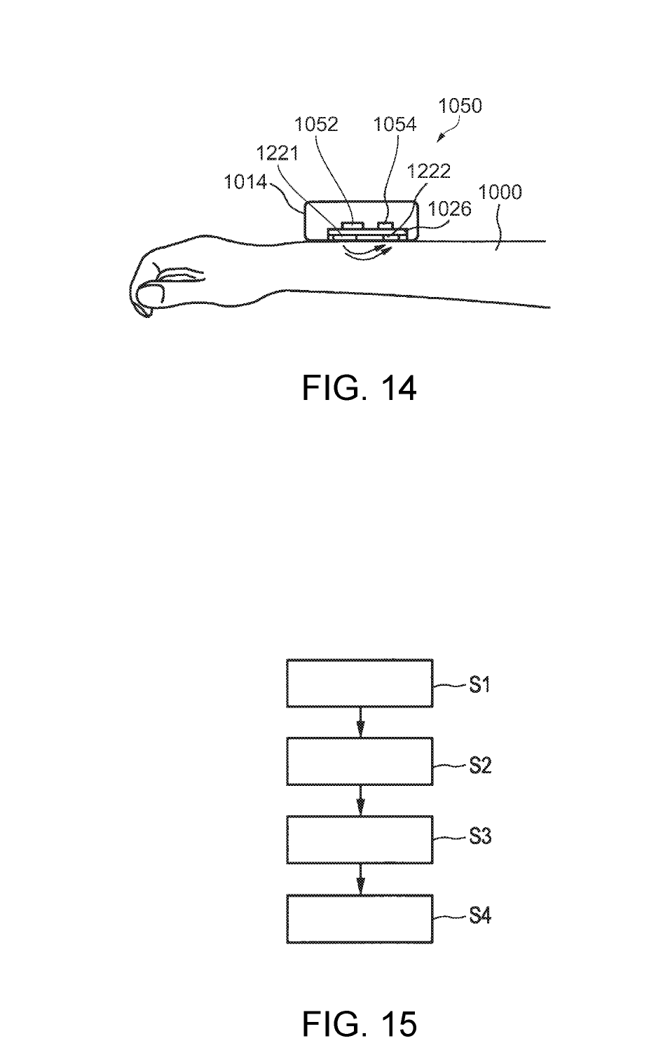

A heart rate monitoring apparatus 1050 as the biological information measuring apparatus according to the fifth embodiment will be described with reference to FIG. 14. FIG. 14 is a cross-sectional view illustrating a heart rate monitoring apparatus as the biological information measuring apparatus according to the fifth embodiment. Meanwhile, although not shown in FIG. 14, the heart rate monitoring apparatus 1050 as the biological information measuring apparatus according to the fifth embodiment is worn on a user's arm by a fixation portion such as a band, similar to the first embodiment described above.

The heart rate monitoring apparatus 1050 as the biological information measuring apparatus according to the fifth embodiment includes the above-mentioned additional electronic apparatuses (for example, a processor 1052 and a driver 1054). An external electric connection terminal (not shown) is not disposed on a carrier 1026 which is the same as that on which sensor elements (light emitting element 1221 as a light emitting unit and a light receiving element 1222 as a light receiving unit) are disposed. In other words, the additional electronic apparatuses are disposed on a carrier different from the carrier on which the sensor elements are disposed, or a substrate. With such a configuration, it is possible to mount necessary additional electronic apparatuses on the heart rate monitoring apparatus 1050 while maintaining a satisfactory contact between the skin and the sensor elements (light emitting element 1221 and the light receiving element 1222). For example, the external electric connection terminal can be disposed on the side surface of the carrier 1026.

As described above, different types of sensors can be used in the biological information measuring apparatus according to the invention. For example, when the light receiving element 1222 mentioned above is an electric sensor, two skin conductance electrodes (for example, sensor elements (the light emitting element 1221 and the light receiving element 1222 which are illustrated in FIG. 11)) which come into contact with the skin of a user and measure the conductivity of the user are covered with the skin. Meanwhile, two or more types of sensors can be used in such a type of biological information measuring apparatus, and the number of sensor elements does not matter.

In the second to fifth embodiments, a flow chart of a method of manufacturing the proposed biological information measuring apparatus that measures a physiologic parameter is illustrated in FIG. 15.

In first step S1, the sensor 1022 including at least two sensor elements (the light emitting element 1221 and the light receiving element 1222) for detecting a sensor signal is disposed on the carrier 1026. In second step S2, an electrical contact between the sensor elements is formed in the carrier 1026. In third step S3, one or more frames 1041 and 1042 are formed on the carrier 1026 in the vicinity of the sensor 1022 and/or the individual sensor elements (the light emitting element 1221 and the light receiving element 1222). In fourth step S4, the insulating material 1032 is injected into and filled in regions surrounded by the respective frames 1041 and 1042 so as not to cover the upper surfaces 1221a and 1222a of the sensor elements (the light emitting element 1221 and the light receiving element 1222) which are provided on the carrier 1026.

According to the second to fifth embodiments described above, a method of protecting an electrical contact that does not exert a bad influence on the performance of the biological information measuring apparatus is proposed. The biological information measuring apparatus is formed by such a method as that in which the performance of a sensor is maintained. For example, at least one of the frames 1041 and 1043 prevents the position of the sensor with respect to the skin from being shifted. Further, at least one of the frames 1041 and 1043 can help emitted direct light to be prevented from being input to the light receiving element 1222. It is preferable that the heights of the frames 1041 and 1043, facing the light receiving element 1222, in the vicinity of the respective light emitting elements 1221 and 1223 have to be smaller than the heights of the upper surfaces 1221a and 1223a of the respective light emitting elements 1221 and 1223. In addition, the frame 1042 in the vicinity of the light receiving element 1222 may be higher than the upper surface 1222a of the light receiving element 1222.

Also in the biological information measuring apparatuses according to the second to fifth embodiments described above, it is possible to apply a configuration of a dimensional relationship between the light emitting unit and the light receiving unit described in the first embodiment, for example, circumference lengths of the outer circumferences of the light emitting unit and the light receiving unit, the areas of the light emitting unit and the light receiving unit, a dimension of an interval between the light emitting unit and the light receiving unit, and the like. With such a configuration, it is possible to obtain the same effects as those in the first embodiment.

### Sixth Embodiment

The biological information measuring apparatuses of the first to fifth embodiments described above may include various types of sensors such as, a strain gauge, a thermometer, a clinical thermometer, an acceleration sensor, a gyro sensor, a piezoelectric sensor, a pressure sensor, a sphygmomanometer, an electrochemical sensor, a global positioning system (GPS), and a vibrometer. The biological information measuring apparatuses include these sensors, and thus it is possible to derive information regarding a personal physiological state on the basis of data indicating one or one or more physiological parameters, such as heartbeat, pulse, a variation between pulsations, an elektrokardiogram (EKG), an electrocardiogram (ECG), a respiration rate, a skin temperature, a body temperature, a body heat flow, a galvanic skin response, a galvanic skin reflex (GSR), an electromyogram (EMG), an electroencephalogram (EEG), an electrooculography (EOG), blood pressure, body fat, a hydration level, an activity level, a body motion, oxygen consumption, glucose, a blood glucose level, muscle mass, pressure applied to a muscle, pressure applied to a bone, ultraviolet absorption, a sleep state, a physical condition, a stress state, and a posture (for example, lying, standing upright, and sitting). In addition, values obtained by the various types of sensors are transmitted to, for example, a portable communication terminal such as a smartphone, a mobile phone, or a feature phone, or an information processing terminal such as a computer or a tablet computer, so that the portable communication terminal or the information processing terminal may execute the arithmetic processing of the physiological parameters.

A user inputs his or her own profile to the biological information measuring apparatus, the portable communication terminal, or the information processing terminal before measuring biological information. Thereby, the user can receive user's unique characteristic information and environmental information which are required to be coped with, in order to maximize a possibility of a recommended healthy lifestyle being established and maintained, on the basis of the profile and biological information measurement results. Examples of information to be provided include one or two or more of exercise information such as an exercise type, an exercise strength, and an exercise time, meal information such as a meal time, the amount of meal, recommended intake ingredients and intake menus, and intake ingredients and intake menus that should be avoided, life support information such as a sleep time, the depth of sleep, the quality of sleep, a wake-up time, a landing time, a working time, stress information, consumed calories, intake calories, and calorie balance, physical information such as basal metabolism, the amount of body fat, a body fat percentage, and muscle mass, medication information, supplement intake information, and medical information.

Examples of the user's own profile input in advance include one or two or more of the age, the date of birth, the sex, hobbies, an occupation type, a blood type, a past sports history, an activity level, meal, the regularity of sleep, the regularity of bowel habit, situation adaptability, durability, responsiveness, the strength of reaction, user's personality such as a temper, a user's self-independence level, independent formation, self-management, sociability, a memory and an academic attainment ability, a user's awakening level, a perception speed, an ability to avoid attention alienation factors, user's attention including an awakening state and a self-supervision ability, an attention continuance ability, the weight, the height, blood pressure, a user's health state, medical examination results by a doctor, the date of a medical examination by a doctor, the presence or absence of a contact between a doctor and a health care person, medicines and supplements that are currently taken, the presence or absence of an allergy, an allergy history, the current allergy symptoms, an opinion of behavior pertaining to health, a user's disease history, a user's operation history, a family medical history, a social phenomenon, such as a divorce or unemployment, which is required to be adjusted by an individual, conviction pertaining to a user's health priority, a sense of values, an ability to change behavior, a phenomenon considered to be a cause of the stress of life, a stress management method, the degree of user's own consciousness, the degree of user's empathy, the degree of user's authority trans fer, user's pride, user's exercise, a sleep state, a relaxed state, the current routine of daily activity, the personality of an important person (for example, a spouse, a friend, a colleague, or a superior officer), and a user's way to catch whether a conflict that disturbs a healthy lifestyle or contributes to stress is present in a relationship with an important person.

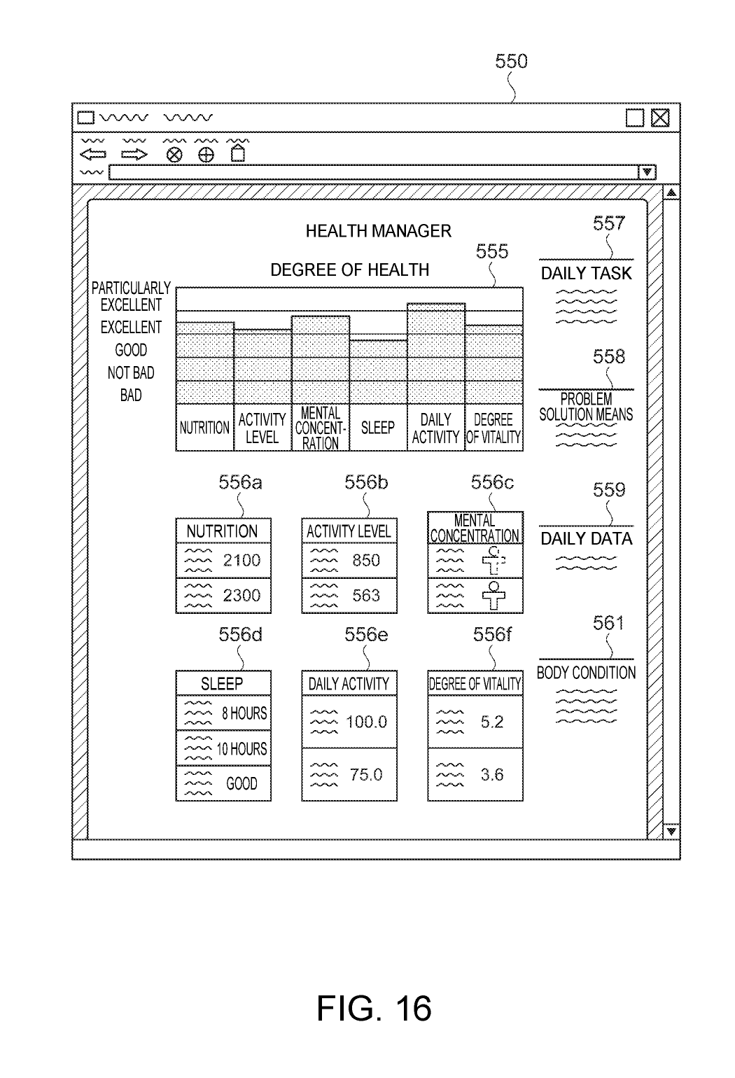

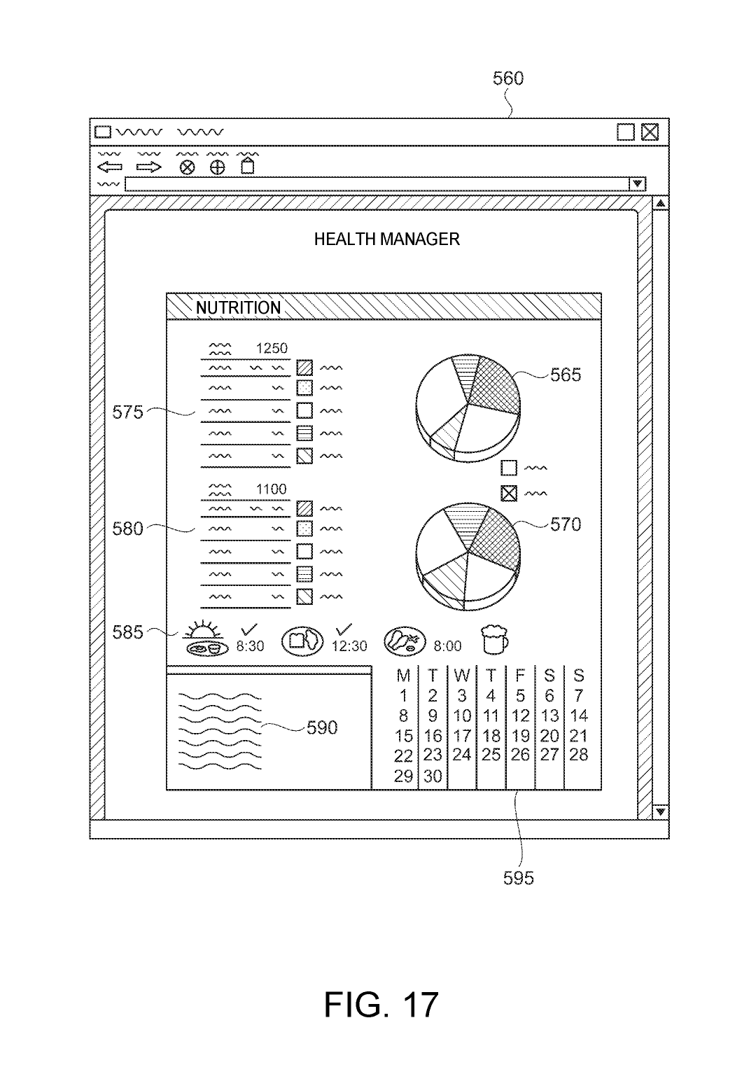

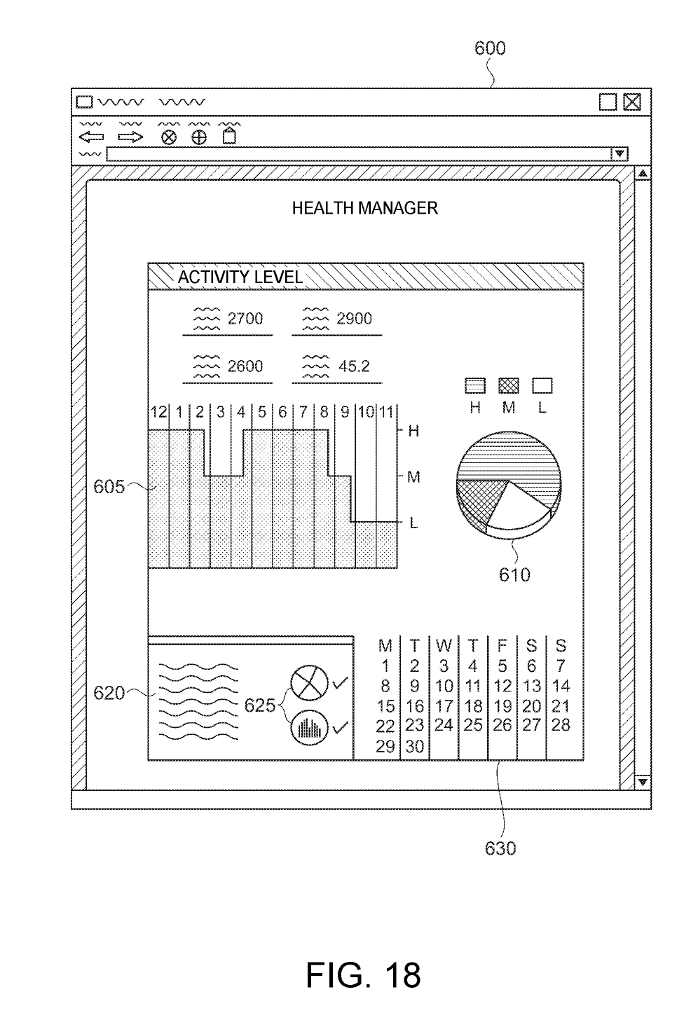

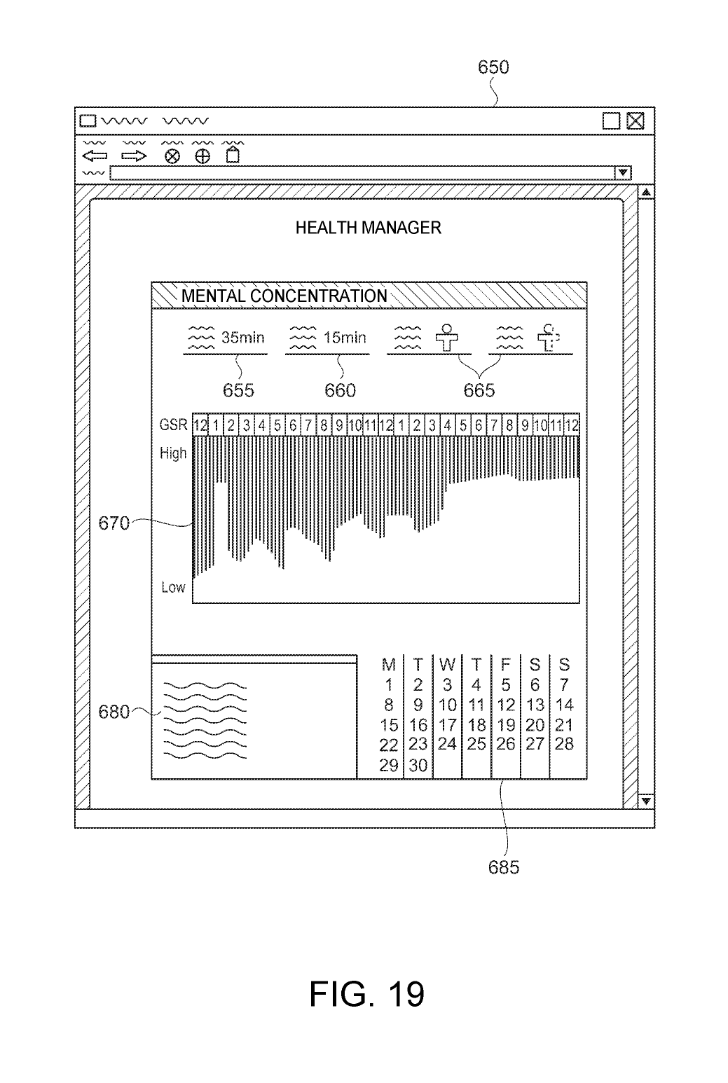

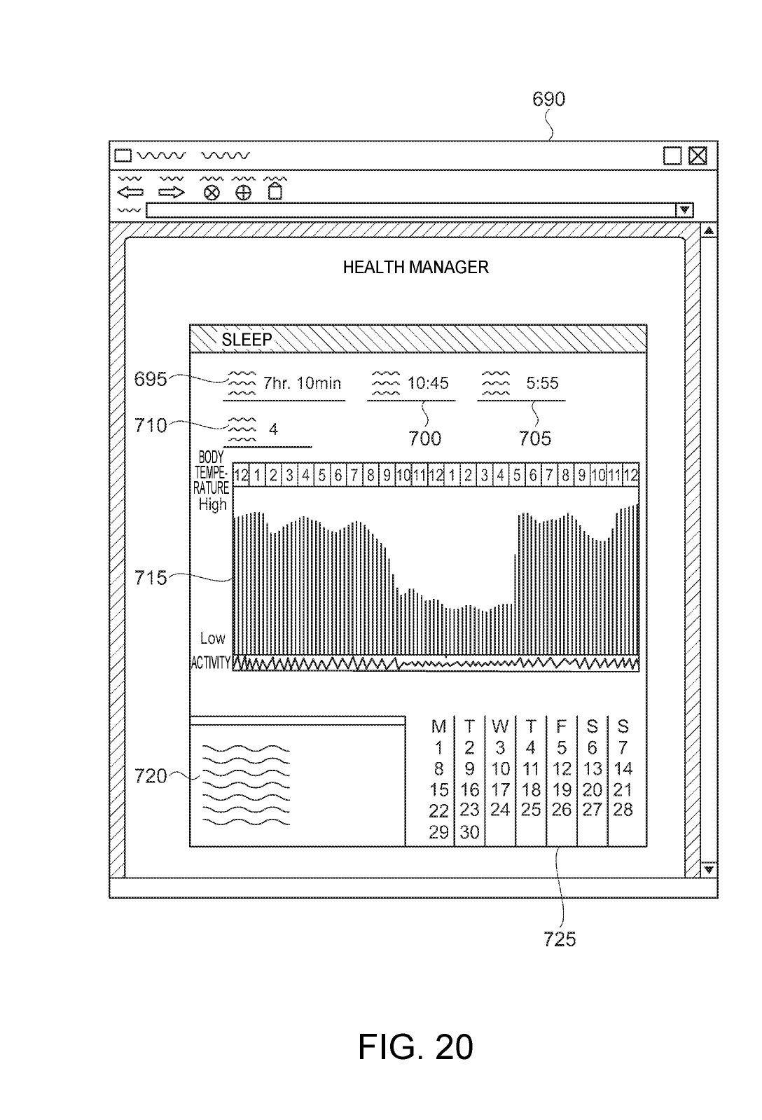

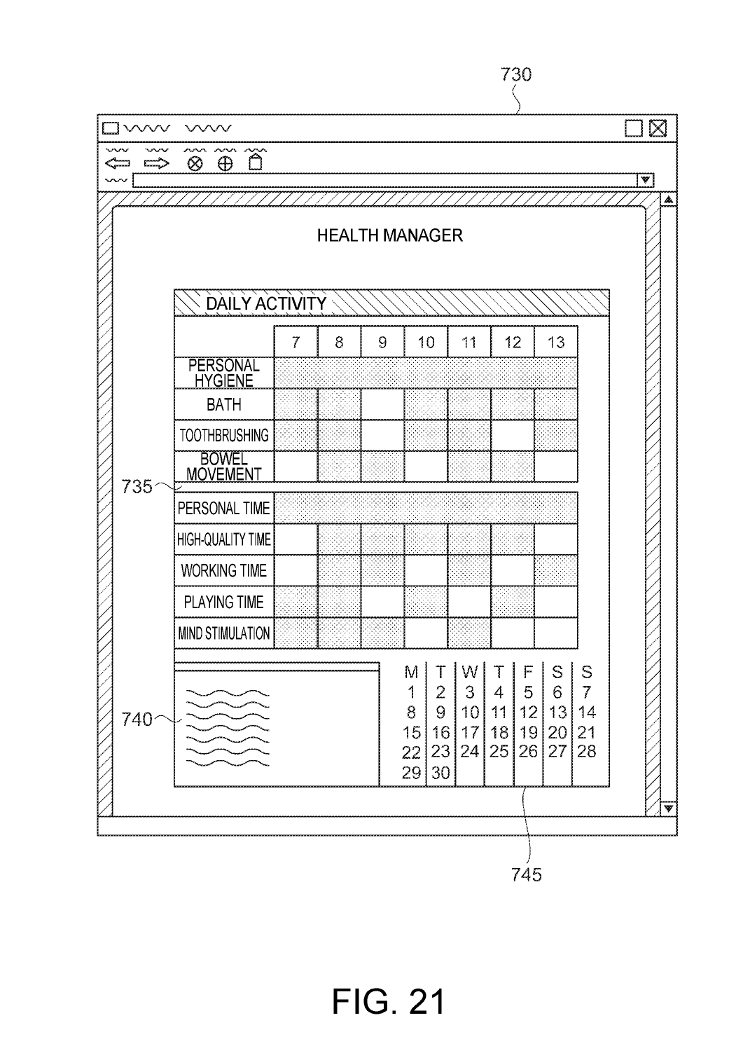

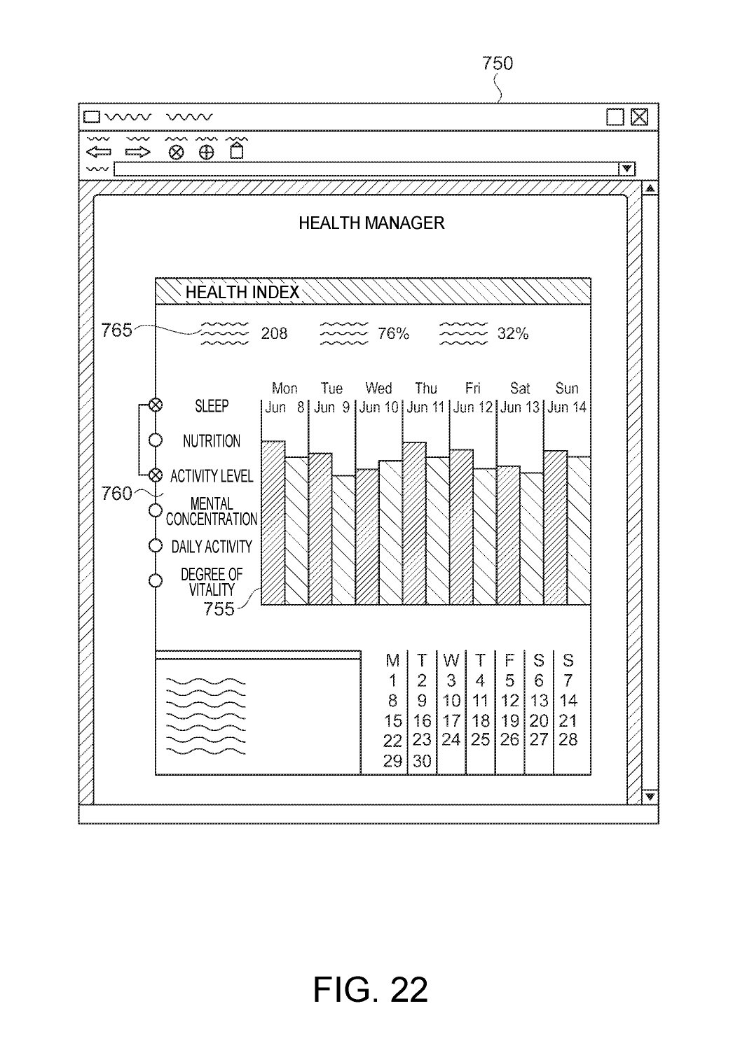

Here, reference will be made to FIGS. 16 to 22 to describe a biological information measuring apparatus according to a sixth embodiment which is capable of receiving user's unique characteristic information and environmental information which are required to be coped with, in order to maximize a possibility of a recommended healthy lifestyle being established and maintained. FIG. 16 is a schematic diagram illustrating a web page serving as a starting point of a health manager in the biological information measuring apparatus of the sixth embodiment. FIG. 17 is a diagram illustrating an example of a nutrition web page, and FIG. 18 is a diagram illustrating an example of an activity level web page. In addition, FIG. 19 is a diagram illustrating an example of a mental concentration web page, and FIG. 20 is a diagram illustrating an example of a sleep web page. In addition, FIG. 21 is a diagram illustrating an example of a daily activity web page, and FIG. 22 is a diagram illustrating an example of a health degree web page.

Although not shown in the drawing, the biological information measuring apparatus according to the sixth embodiment includes, for example, a sensor device which is connected to a microprocessor. In the biological information measuring apparatus according to the sixth embodiment, pieces of data regarding various life activity items which are finally transmitted to a monitor unit and stored, and personal data or living information which is input by a user from a website maintained by the monitor unit are processed by the microprocessor and are provided as biological information. Hereinafter, a specific example will be described.

A user has access to a health manager for the user through a web page, application software, and other communication media. FIG. 16 illustrates a web page 550 serving as a starting point of the health manager, as an example. In the web page 550 of the health manager shown in FIG. 16, various pieces of data are provided to a user. The provided data is one or more pieces of data of, for example, (1) data indicating various physiological parameters based on values measured by various sensor devices, (2) data derived from data indicating various physiological parameters, and (3) data indicating various context parameters generated by the sensor device and data input by the user.

Analysis state data has features that a certain utility or algorithm is used in order to perform conversion into (1) data indicating various physiological parameters acquired by the sensor device, (2) data derived from various physiological parameters, (3) the degree of health obtained by calculating one or more pieces of data of data indicating various context parameters acquired by the sensor device and data input by the user, (4) the degree of good health and a lifestyle index, and the like. For example, it is possible to calculate the amounts of calories, protein, fat, carbohydrates, and certain vitamin on the basis of data input by the user in relation to food taken. In addition, as another example, it is possible to provide indexes of stress levels over a desired period of time to the user by using a skin temperature, a heart rate, a respiration rate, a heat flow and/or a GSR. As still another example, it is possible to provide indexes of sleep patterns over a desired period of time to the user by using a skin temperature, a heat flow, a variation between pulsations, a heart rate, pulse, a respiration rate, a central body temperature, a galvanic skin response, an EMG, an EEG, an EOG, blood pressure, oxygen consumption, ambient sounds, and body motion detected by a device such as an accelerometer.

In the web page 550 illustrated in FIG. 16, a health index 555 as the degree of health is displayed. The health index 555 is a graphic utility for measuring the degree of achievement of user's results and a recommended healthy daily task and giving feedback to member users. In this manner, the health index 555 indicates health states and progress conditions of action pertaining to health maintenance of the member users. The health index 555 includes six categories regarding the health and lifestyle of a user, that is, nutrition, an activity level, mental concentration, sleep, daily activity, and the degree of vitality (overall impression). The category of “nutrition” pertains to information regarding what, when, and how much the person (user) has eaten and taken. The category of “activity level” pertains to the amount of exercise regarding how much the person has moved around. The category of “mental concentration” pertains to the quality (ability) of the activity for making the person (user) set to be in a relaxed state in a state where the mind of the person is in a highly concentrated state, and to a period of time for which the person concentrates on the activity. The category of “sleep” pertains to the quality and amount of sleep of the person (user). The category of “daily activity” pertains to matters that have to be performed every day by the person (user) and to health risks that the person meets with. The category of “the degree of vitality (impression)” pertains to a general way to catch whether being in a good mood on a certain day. Preferably, each of the categories includes a level display or a bar graph indicating how many results the user has attained on a scale varying between “bad” and “good”.

When each member user terminates the above-mentioned initial examination, a profile for providing a user's own characteristics and a summary of a living environment to the user is created, and recommended healthy daily tasks and/or targets are presented. The recommended healthy daily tasks include any combination in specific pieces of advices regarding appropriate nutrition, exercise, mental concentration, and user's daily activity (life). A model schedule or the like may be presented as a guide indicating how to take activity items pertaining to the recommended healthy daily tasks in the user's life. The user is regularly subjected to the examination, and practices the above-mentioned items accordingly on the basis of the results thereof.

The category of “nutrition” is calculated from both data input by a user and data sensed by a sensor device. The data input by the user includes the times for breakfast, lunch, and dinner, and any snack and the eating and drinking times thereof, and food to be eaten and drunk, supplements such as vitamin, and water or another liquid (drinking water or liquid food) which is drunk during a time which is selected in advance. A central monitoring unit calculates consumed calories or well-known nutritional values such as the contents of protein, fat, carbohydrates, vitamin, and the like, on the basis of the data and stored data regarding known characteristics of various articles of food.

In the category of “nutrition”, a recommended healthy daily task can be determined on the basis of the bar graph indicating the nutrition of the health index 555. The recommended healthy daily task can be adjusted on the basis of information such as the sex, age, and height/weight of a user. Meanwhile, a user or a representative of the user can set a target of certain nutrition pertaining to the amount of calories consumed every day, the amount of nutriments such as protein, fiber, fat, and carbohydrates, the amount of water, and ratios thereof to the total intake. Parameters used for the calculation of the bar graph include the number of meals for one day, the amount of water consumed, and the type and amount of food eaten every day which are input by a user.

Nutritional information is presented to a user by a nutrition web page 560 as illustrated in FIG. 17. It is preferable that the nutrition web page 560 includes nutrition numerical charts 565 and 570 that are pie charts showing actual and target numerical values of nutrition, and nutrition intake charts 575 and 580 showing an actual total nutrition intake amount and a target total nutrition intake amount. In the nutrition numerical charts 565 and 570, it is preferable that items such as carbohydrates, protein, and fat are expressed by percentage. In the nutrition intake charts 575 and 580, it is preferable that a total value and a target value of calories are expressed by being divided into ingredients such as fat, carbohydrates, protein, and vitamin. The web page 560 includes a history 585 indicating the times when food and water are consumed, a hyperlink 590 that allows a user to be able to directly check a news story pertaining to nutrition, advice for improving a daily task pertaining to nutrition, and any related advertisement on a network, and a calendar 595 in which an application period and the like can be selected. Items indicated by the hyperlink 590 can be selected on the basis of information learned from an individual through examination, and the individual's results measured by the health index.

The category of “activity level” in the health index 555 is designed so as to support a user's check regarding when and how the user had activity (moved) on that day, and the like, and both data input by the user and data sensed by the sensor device are used. The data input by the user includes details pertaining to the user's daily activity such as, for example, doing work at the desk from 8 a.m. to 5 p.m. and taking an aerobic lesson from 6 p.m. to 7 p.m. The related data sensed by the sensor device includes a heart rate, an exercise sensed by a device such as an accelerometer, a heat flow, a respiration rate, the amount of calories consumed, a GSR, and a water supply level, and these can be taken out by the sensor device or the central monitoring unit. The amount of calories consumed can be calculated by various methods such as multiplication of the type of exercise which is input by the user and the duration of exercise which is input by the user, multiplication of the sensed exercise, an exercise time, and a filter constant, or multiplication of the sensed heat flow, the time, and a filter constant.

In the category of “activity level”, a recommended healthy daily task can be determined on the basis of the bar graph indicating the activity level of the health index 555. The recommended healthy daily task includes a minimum target calories consumed by the activity, and the like. Meanwhile, the minimum target calories can be set on the basis of information such as the sex, age, height, and weight of a user. Parameters used for the calculation of the bar graph includes a time input by the user and/or a time sensed by the sensor device which are times spent for various types of exercises or an energetic lifestyle activity, and the amount of calories burned over an energy consumption parameter which is calculated in advance.

Information regarding the activity (movement) of an individual user is presented to the user by an activity level web page 600 illustrated in FIG. 18. The activity level web page 600 includes an activity degree graph 605, having a bar graph shape, which shows the user's activity monitored according to three categories, that is, “high”, “medium”, and “low” that are classified with respect to a predetermined unit time. An activity percentage chart 610 having a pie chart shape can be presented in order to express a percentage for a predetermined period of time such as, for example, one day which is spent in each of the categories by the user. In addition, the activity level web page 600 may include a calory display (not shown) for displaying items such as a total amount of calories burned, a target value of daily burned calories, a total value of calories taken, and an aerobic exercise time. The activity level web page 600 includes at least one hyperlink 620 in order to allow the user to be able to directly check a related news story, advice for improving a daily task pertaining to an activity level, and a related advertisement on a network.

The activity level web page 600 can be viewed in various formats, and can be configured such that a user can select a bar graph, a pie chart, or both the graph and the chart and the selection can be performed by an activity level check box 625. An activity level calendar 630 is provided so that an application period and the like can be selected. Items indicated by the hyperlink 620 can be selected on the basis of information extracted from an individual through examination, and the results measured by the health index.

The category of “mental concentration” in the health index 555 is designed so as to support a user's monitoring of parameters pertaining to a time when the activity for allowing the user's body to reach a deep relaxed state while concentrating his or her mind is performed, and is based on both data input by the user and data sensed by the sensor device. In detail, the user can input a starting time and a termination time of a relaxation activity such as yoga or meditation. The quality of these activity items determined by the depth of mental concentration can be measured by monitoring parameters including a skin temperature, a heart rate, a respiration rate, and a heat flow which are sensed by the sensor device. It is also possible to use a variation in the percentage of a GSR obtained by either of the sensor device or the central monitoring unit.

In the category of “mental concentration”, a recommended healthy daily task can be determined on the basis of the bar graph indicating the activity level of the mental concentration in the health index 555. The recommended healthy daily task is displayed inclusive of daily joining in the activity of deeply relaxing a body while making mind set to be in a highly concentrated state. Parameters used for the calculation of the bar graph include the length of time spent for the mental concentration activity, the depth of the mental concentration activity, or a variation in the percentage of a skin temperature, a heart rate, a respiration rate, a heat flow, or a GSR which is sensed by the sensor device from a base line indicating quality.

Information regarding time spent for an action of deeply looking back oneself (introspection) and for mental concentration activity such as deep relaxation of a body is presented to a user by a mental concentration web page 650 illustrated in FIG. 19. Meanwhile, the mental concentration activity may be referred to as a session. The mental concentration web page 650 includes a time 655 spent for the session, a target time 660, comparison portions 665 indicating a target value of the depth of mental concentration and an actual value, and a histogram 670 indicating the overall stress level which is derived from a skin temperature, a heart rate, a respiration rate, a heat flow, and/or a GSR.

In the comparison portion 665, the contour of a human indicating a target mental concentration state is shown by a solid line, and the contour of a human indicating an actual mental concentration state varies between a blurred state (shown by a dashed line in FIG. 19) and a solid line in accordance with the level of mental concentration. In addition, the preferable mental concentration web page 650 includes a hyperlink 680 that allows a user to be able to directly check a related news story, advice for improving a daily task pertaining to mental concentration, and a related advertisement on a network, and a calendar 685 in which an application period can be selected. Items indicated by the hyperlink 680 can be selected on the basis of results measured by information learned from an individual through examination, and the results measured by the health index.

The category of “sleep” in the health index 555 is designed so as to be able to support a user's monitoring of a sleep pattern and the quality of sleep. This category is intended to help a user to learn the importance of sleep in a healthy lifestyle and the relation of sleep to a daily cycle which is an ordinary daily variation in the function of the body. The category of “sleep” is based on both data input by the user and data sensed by the sensor device. The data input by the user between related time intervals includes ranks of a sleep-onset time and a wake-up time (sleep time) of the user and the quality of sleep. The related data obtained by the sensor device includes a skin temperature (body temperature), a heat flow, a variation between pulsations, a heart rate, a pulse rate, a respiration rate, a central body temperature, a galvanic skin response, an EMG, an EEG, an EOG, blood pressure, and oxygen consumption. In addition, ambient sounds and body motion which is detected by a device such as an accelerometer also have relevance. Thereafter, a sleep-onset time, a wake-up time, the interruption of sleep, the quality of sleep, the depth of sleep, and the like can be calculated and derived using the data.

The bar graph showing the sleep in the health index 555 displays a healthy daily task including the securing of a preferable nightly minimum sleep time, a predictable bedtime, and a wake-up time. Specific parameters enabling the calculation of the bar graph include a daily sleep time and a wake-up time which are sensed by the sensor device or input by the user, and the quality of sleep which is graded by the user or derived from another data.

Information regarding the sleep is presented to a user by a sleep web page 690 illustrated in FIG. 20. The sleep web page 690 includes a sleep time display 695 based on either of data from the sensor device or data input by the user, a user bedtime display 700, and a wake-up time display 705. Meanwhile, the quality of sleep which is input by the user can be displayed using a sleep quality rank 710. In addition, when a display exceeding a time interval for one day is performed in the sleep web page 690, the sleep time display 695 can be displayed as a cumulative value, and the bedtime display 700, the wake-up time display 705, and the sleep quality rank 710 can be calculated and displayed as average values. In addition, the sleep web page 690 also includes a sleep graph 715 selectable by a user who calculates and displays one sleep-related parameter during a predetermined time interval. FIG. 20 illustrates a variation in a heat flow (body temperature) for one day. The heat flow tends to be reduced while asleep and to be increased while awake. It is possible to obtain a biorhythm of the person from the information.

In addition, the sleep graph 715 graphs data from an accelerometer embedded in the sensor device that monitors body motion. In addition, the sleep web page 690 can include a hyperlink 720 that allows a user to be able to directly check a news story pertaining to sleep, advice for improving a daily task pertaining to sleep, and a related advertisement on a network, and a sleep calendar 725 for selecting a related time interval. Items indicated by the hyperlink 720 can be particularly selected on the basis of information learned from an individual in examination, and results measured by the health index.

The category of “daily activity” in the health index 555 is designed so as to be able to support a user's monitoring of a certain activity, pertaining to health or safety, and risk, and is completely based on data input by a user. The category of “daily activity” pertaining to activity in a daily life includes four categories which are subordinate concepts. Specifically, the category is classified into (1) an item pertaining to personal hygiene which enables a user's monitoring of dental care using a toothbrush or floss or activity such as taking a shower, (2) an item pertaining to health maintenance which enables tracing of whether a user is taking medicine or a supplement as prescribed, and enables a user's monitoring of the consumption of cigarettes or alcohol, and the like, (3) an item pertaining to personal time which enables a user's monitoring of time or leisure, which is spent with the user's family or friend, and mental concentration activity, and (4) an item pertaining to responsibility which enables a user's monitoring of work, such as household chores, and household activity.

In the category of “daily activity”, it is preferable that the bar graph indicating the “daily activity” in the health index 555 displays the following recommended healthy daily tasks. As an example of a daily task pertaining to the personal hygiene, it is preferable that a user takes a shower or takes a bath every day, keeps his or her teeth clean by using a toothbrush or floss every day, and has regular bowel movements. In addition, as an example of a daily task pertaining to the health maintenance, it is preferable that a user takes medicine, vitamin pills, and/or supplements, does not smoke, drinks in moderation, and monitors his or her health every day by a health manager. As an example of a daily task pertaining to the personal time, it is preferable that a user makes at least predetermined time every day in order to spend the time with his or her family, and/or spends high-quality time with his or her friend, reduces time for work, takes time for leisure or play, and performs activity using his or her brain. As an example of a daily task pertaining to the responsibility, it is preferable that a user does household chores, is not late for work, and keeps a promise. The bar graph is determined by information input by a user, and/or is calculated on the basis of the degree to which the user completes activity listed up every day.

Pieces of information regarding these activity items are presented to a user by a daily activity web page 730 illustrated in FIG. 21. An activity chart 735 in the daily activity web page 730 shows whether a user has executed necessary activity by the daily task. In the activity chart 735, one or more of the subordinate concepts can be selected. In the activity chart 735, a box which is colored or shaded indicates that a user has executed necessary activity, and a box which is not colored or shaded indicates that the user has not executed the activity. The activity chart 735 can be created at a selectable time interval and can be viewed. FIG. 21 illustrates the categories of personal hygiene and personal time in a specific week as an example. Further, the daily activity web page 730 may include a hyperlink 740 that allows a user to be able to directly check a related news story, advice for improving a daily task pertaining to activity in a daily life, and a related advertisement on a network, and a daily activity calendar 745 for selecting a related time interval. Items indicated by the hyperlink 740 can be selected on the basis of information learned from an individual in examination, and results determined by the health index.

The category “the degree of vitality” in the health index 555 is designed so as to enable a user's monitoring of recognition of whether being in good spirits on a specific day, and is based on essentially subjective grade information which is directly input by the user. The user performs ranking using scales of, preferably, 1 to 5 with respect to the following nine areas, that is, (1) mental keenness, (2) the degree of mental and psychological happiness, (3) an energy level, (4) a capacity for stresses of life, (5) the degree of being concerned about appearances, (6) the degree of physical happiness, (7) self-control, (8) a motive, and (9) comfort by a relationship with others. These degrees (grades) are averaged to be used for the calculation of the bar graph of the health index 555.

FIG. 22 illustrates a vitality degree web page 750. The vitality degree web page 750 allows a user to be able to check the degree of vitality during a time interval, selectable by the user, which includes continuous or discontinuous arbitrary days. Meanwhile, in the example illustrated in FIG. 22, the degree of vitality is displayed as a health index. In the vitality degree web page 750, a user can perform selection for checking a vitality degree bar graph 755 with respect to one category or can compare the vitality degree bar graphs 755 in parallel with respect to two or more categories by using the vitality degree selection box 760. For example, the user may set only a bar graph for sleep to be in an operation state in order to check whether the overall grade of sleep has been improved compared to the previous month, or may compare the grade of sleep with the grade of an activity level corresponding thereto and evaluates the grades by simultaneously displaying the sleep and the activity level and may check whether there is some correlation between the days. The grade of nutrition and the grade of the degree of vitality may be displayed for a predetermined time interval so that it is checked whether there is some correlation between a daily dietary habit, a dietary habit during the interval, and the degree of vitality. FIG. 22 illustrates comparison between sleep and an activity level during a week from June 8 to June 14 using bar graphs, as an example for description. In addition, the vitality degree web page 750 also includes a tracing calculator 765 that displays access information, such as the sum of days in which a user has logged on and used the health manager, the proportion of days in which the user has used the health manager since admission, and the proportion of hours for which the user has used the sensor device in order to collect data, and statistics.

An example of the web page 550 serving as a starting point of the health manager illustrated in FIG. 16 includes summaries 556a to 556f of a plurality of categories, selectable by a user, which correspond to the categories of the health index 555 as the degree of health. Each of the summaries 556a to 556f of the respective categories presents a sub set of data which is selected in advance with respect to the corresponding category and is filtered. The summary 556a of the category of nutrition indicates a daily target value and an actual value of a caloric intake. The summary 556b of the category of activity level indicates a daily target value and an actual value of the amount of calories burned. The summary 556c of the category of mental concentration indicates a target value and an actual value of the depth of mental concentration. The summary 556d of the category of sleep indicates a target sleep time, an actual sleep time, and the grade of the quality of sleep. The summary 556e of the category of daily activity displays a target point and an actual point based on a ratio of completed activity to a recommended healthy daily task (daily activity). The summary 556f of the category of the degree of vitality indicates a target grade and an actual grade of the degree of health of the day.

In addition, the web page 550 may also include a hyperlink (not shown) to a news story, comments (not shown) to a user based on a tendency such as malnutrition which is checked by the first examination, and a signal (not shown). The web page may also include a daily task portion 557 that provides information to a user every day. As comments of the daily task portion 557, for example, a water intake required every day, advice for specific means for enabling the intake of water, and the like can be displayed. In addition, the web page 550 may include a problem solution section 558 that actively evaluates a user's results in each category of the health index 555 and presents advice for improvement. For example, when a user's sleep level is “low” by a system and it is suggested that the user has insomnia, the problem solution section 558 can advise a method for improving sleep. In addition, the problem solution section 558 may include the user's question regarding an improvement in results. In addition, the web page 550 may include a daily data section 559 that starts up an input dialogue box. The user can easily input various pieces of data required by the health manager, using the input dialogue box. As known in the art, the input of data can be selectively performed between the input in a list presented in advance and the input in a general free text format. In addition, the web page 550 may include a body condition section 561 that gives information regarding life symptoms such as the height and weight of a user, a body measurement value, a BMI, a heart rate, blood pressure, or any physiological parameter.

### Modification Example of Light Receiving Unit

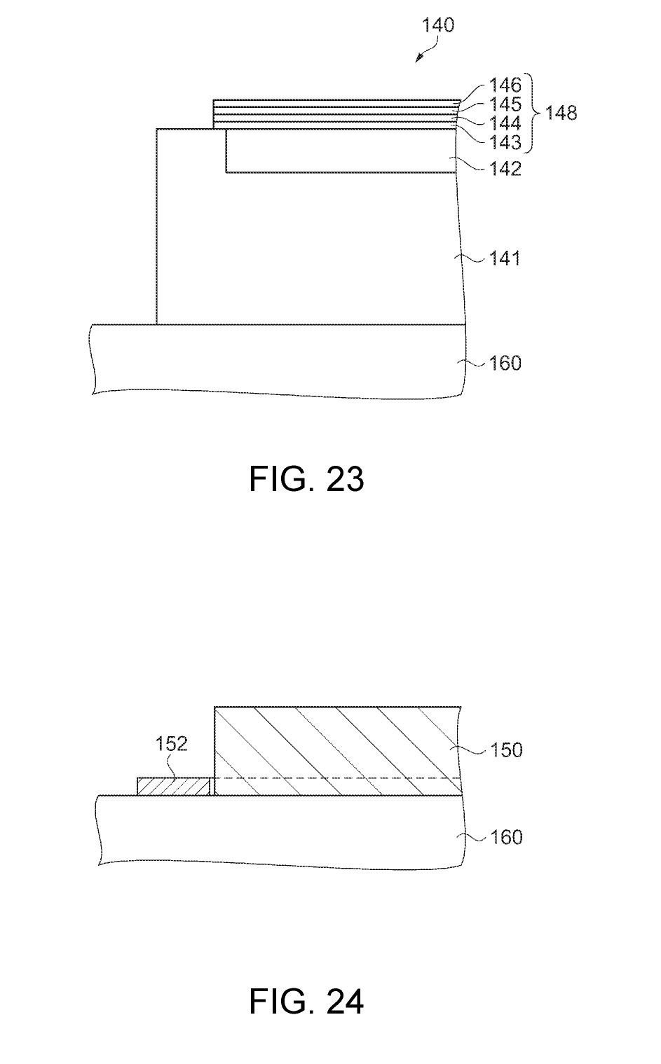

Here, a modification example of the light receiving unit 140 mentioned above will be described with reference to FIG. 23. FIG. 23 is a partial cross-sectional view illustrating a modification example of a light receiving unit. As illustrated in FIG. 23, a light receiving unit 140 mounted on a substrate 160 (sensor substrate) can be realized by a diode element 142 of a PN junction which is formed on a semiconductor substrate 141, and the like. In this case, an angle limiting filter for narrowing a light reception angle or a wavelength limiting filter (optical filter film) 148 that limits a wavelength of light incident on a light receiving element may be formed on the diode element 142. Meanwhile, for example, the wavelength limiting filter (optical filter film) 148 can be configured such that a first oxide film 143, a first nitride film 144, a second oxide film 145, and a second nitride film 146 are formed from the diode element 142 side in this order.

With such a configuration, it is possible to provide the wavelength limiting filter (optical filter film) 148 in a smaller region and to provide a smaller-sized biological information measuring module and biological information measuring apparatus.

### Modification Example of Light Emitting Unit

Next, a modification example of the light emitting unit 150 mentioned above will be described with reference to FIG. 24. FIG. 24 is a partial cross-sectional view illustrating a modification example of a light emitting unit. As illustrated in FIG. 24, a reflective functional layer 152 that reflects light emitted in a peripheral direction from a light emitting unit 150 is provided in the vicinity of the light emitting unit 150 mounted on a substrate 160 (sensor substrate). Meanwhile, the reflective functional layer 152 may be provided so as to surround the vicinity of the light emitting unit 150 over the whole periphery or may be provided in at least a portion of the vicinity of the light emitting unit 150 in a plan view seen from the upper surface side of the substrate 160.

With such a configuration, light emitted in a peripheral direction of the light emitting unit 150 can be made to be reflected by a reflective functional layer 152 and to be directed to a measurement object. Thereby, it is possible to increase the intensity (light emission intensity) of light directed to the measurement object, and to improve and stabilize the measurement accuracy of biological information.

Meanwhile, embodiments of the invention have been described above in detail, but those skilled in the art may easily understand that many variations are conceivable to the extent that they do not substantially depart from the novel items and effects of the invention. Therefore, such variations all fall within the scope of the invention. For example, a term described at least once in the specification or the drawings with a different term having a broader meaning or the same meaning can be replaced with the different term anywhere in the specification or the drawings. Further, the configuration and action of each of the biological information measuring module, the light detection unit, the biological information measuring apparatus, and the like are not limited to those described in this embodiment of the invention, and a variety of changes can be made thereto.

What is claimed is:

1. A biological information measuring module comprising:
a light emitting unit that emits light to an object; and
a light receiving unit that receives light which is reflected by the object,
wherein a circumference length of the light emitting unit on the outer circumference is equal to or greater than 1.9 mm and equal to or less than 9.5 mm.

2. The biological information measuring module according to claim 1, wherein a circumference length of the light emitting unit on the outer circumference is equal to or greater than 2.5 mm and equal to or less than 8.0 mm.

3. The biological information measuring module according to claim 1, wherein a circumference length of the light emitting unit on the outer circumference is equal to or greater than 3.0 mm and equal to or less than 5.0 mm.

4. A biological information measuring module comprising:
a light emitting unit that emits light to an object; and
a light receiving unit that receives light which is reflected by the object,
wherein a circumference length of the light receiving unit on the outer circumference is equal to or greater than 5.3 mm and equal to or less than 11.7 mm.

5. The biological information measuring module according to claim 4, wherein a circumference length of the light receiving unit on the outer circumference is equal to or greater than 5.8 mm and equal to or less than 11.0 mm.

6. The biological information measuring module according to claim 4, wherein a circumference length of the light receiving unit on the outer circumference is equal to or greater than 6.8 mm and equal to or less than 9.0 mm.

7. A biological information measuring module comprising:
a light emitting unit that emits light to an object; and
a light receiving unit that receives light which is reflected by the object,
wherein an area of the light emitting unit is equal to or greater than 2.5 mm² and equal to or less than 5.0 mm².

8. The biological information measuring module according to claim 7, wherein an area of the light emitting unit is equal to or greater than 3.0 mm² and equal to or less than 4.6 mm².

9. The biological information measuring module according to claim 7, wherein an area of the light emitting unit is equal to or greater than 3.3 mm² and equal to or less than 4.0 mm².

10. A biological information measuring module comprising:
a light emitting unit that emits light to an object; and
a light receiving unit that receives light which is reflected by the object,
wherein an area of the light receiving unit is equal to or greater than 1.7 mm² and equal to or less than 8.5 mm².

11. The biological information measuring module according to claim 10, wherein an area of the light receiving unit is equal to or greater than 2.3 mm² and equal to or less than 6.3 mm².

12. The biological information measuring module according to claim 10, wherein an area of the light receiving unit is equal to or greater than 3.0 mm² and equal to or less than 4.0 mm².

13. The biological information measuring module according to claim 1, wherein a plurality of the light emitting units are provided.

14. The biological information measuring module according to claim 13, wherein the light receiving unit and the plurality of light emitting units are disposed so as to be lined up in a row in a plan view seen from a vertical direction of a light receiving surface of the light receiving unit.

15. The biological information measuring module according to claim 14,
wherein the plurality of light emitting units include a first light emitting unit and a second light emitting unit, and
wherein the light receiving unit is disposed between the first light emitting unit and the second light emitting unit.

16. The biological information measuring module according to claim 14, wherein the plurality of light emitting units are disposed at line symmetrical positions with respect to a virtual line passing through a center of the light receiving unit.

17. The biological information measuring module according to claim 1, wherein a reflective functional layer that reflects light emitted from the light emitting unit is provided in at least a portion of a vicinity of the light emitting unit.

18. The biological information measuring module according to claim 1, wherein an optical filter film is provided in a light receiving region of the light receiving unit.

19. The biological information measuring module according to claim 1, wherein a light shielding unit is provided between the light emitting unit and the light receiving unit.

20. A biological information measuring apparatus comprising the biological information measuring module according to claim 1.
